(19) United States
(12) Patent Application Publication
(10) Pub. No.: US 2023/0089723 A1
(43) Pub. Date: Mar. 23, 2023

(54) AMINO ACID COMPOSITIONS AND METHODS FOR MUSCLE AND MYOTUBE MODULATION

(71) Applicant: AXCELLA HEALTH INC., Cambridge, MA (US)

(72) Inventors: William Comb, Melrose, MA (US); Murat Cokol, Brookline, MA (US); Tripti Kulkarni, Boston, MA (US)

(21) Appl. No.: 17/909,244

(22) PCT Filed: Mar. 5, 2021
(86) PCT No.: PCT/US2021/021167

§ 371 Date: Sep. 2, 2022

Related U.S. Application Data
(60) Provisional application No. 62/986,338, filed on Mar. 6, 2020.

(51) Int. Cl. A61K 31/198; A61K 31/4172; A61K 31/405; A61P 21/06

(52) U.S. Cl. CPC A61K 31/198; A61K 31/4172; A61K 31/405; A61P 21/06

(57) ABSTRACT

This disclosure provides compositions comprising amino acid entities. The disclosure also provides methods for increasing myogenesis, increasing muscle protein synthesis, increasing muscle mass, improving muscle quality or enhancing muscle function comprising administering an effective amount of the compositions to a subject in need thereof.

### CROSS-REFERENCE TO RELATED APPLICATIONS

This application claims priority under 35 U.S.C. § 119(e) to U.S. Provisional Patent Application Ser. No. 62/986,338, filed Mar. 6, 2020, the disclosure of which is hereby incorporated herein by reference in its entirety for all purposes.

### BACKGROUND

There are a number of diseases and disorders associated with the decrease or degenerative loss of muscle mass. Muscle atrophy is associated poor outcomes across a variety of conditions including aging and a number of diverse diseases, such as cancer, AIDS, renal failure, liver disease, and congestive heart failure. Furthermore, disuse of muscles through immobilization (e.g., bed rest) also results in muscle atrophy.

Sarcopenia is a disease characterized by degenerative loss of skeletal muscle mass (typically 0.5-1% loss per year after the age of 25), quality, and strength associated with aging. Sarcopenia is a component of the frailty syndrome. Frailty is a common geriatric syndrome that embodies an elevated risk of catastrophic declines in health and function among older adults. Contributors to frailty can include sarcopenia, osteoporosis, and muscle weakness.

Thus, there is a need to develop therapeutics to enhance muscle function, such as for treating muscle-related disease and disorders.

### SUMMARY

Disclosed herein, at least in part, is a composition comprising at least five different amino acid entities. In some embodiments, the composition is capable of one, two, three, or all of: a) activating mTORC1; b) activating protein synthesis and/or inhibiting protein breakdown; c) improving, e.g., increasing, insulin sensitivity; d) reducing inflammation; e) improving myogenesis or myotube growth; f) improving muscle mass; g) improving muscle function; h) increasing muscle growth; or i) lowering muscle fat.

Accordingly, in one aspect, the disclosure is directed to a composition comprising: a) a leucine (L)-amino acid entity; b) an isoleucine (I)-amino acid entity; c) a valine (V)-amino acid entity; d) an aspartic acid (D)-amino acid entity; e) a methionine (M)-amino acid entity; f) a cysteine (C)-amino acid entity; g) an arginine (R)-amino acid entity; h) a histidine (H)-amino acid entity; i) a tyrosine (Y)-amino acid entity; and j) a tryptophan (W)-amino acid entity; provided that: (i) at least one amino acid entity is not provided as a peptide of more than 20 amino acid residues in length; (ii) the amino acid entities of (a)-(j) are selected from Table 2; and (iii) the composition comprises fewer than 20 different amino acid entities.

In another aspect, the disclosure is directed to a composition comprising: a) a leucine (L)-amino acid entity; b) an isoleucine (I)-amino acid entity; c) a valine (V)-amino acid entity; d) a glutamine (Q)-amino acid entity; e) a glutamic acid (E)-amino acid entity; f) an aspartic acid (D)-amino acid entity; g) a glycine (G)-amino acid entity; h) a serine (S)-amino acid entity; i) a methionine (M)-amino acid entity; j) a cysteine (C)-amino acid entity; k) an ornithine (Orn)-amino acid entity; l) an arginine (R)-amino acid entity; m) a histidine (H)-amino acid entity; n) a tyrosine (Y)-amino acid entity; and o) a tryptophan (W)-amino acid entity; provided that: (i) at least one amino acid entity is not provided as a peptide of more than 20 amino acid residues in length; (ii) the amino acid entities of (a)-(o) are selected from Table 2; and (iii) the composition comprises fewer than 20 different amino acid entities.

In another aspect, the disclosure is directed to a composition comprising: a) a leucine (L)-amino acid entity; b) an isoleucine (I)-amino acid entity; c) a valine (V)-amino acid entity; d) a glutamic acid (E)-amino acid entity; e) an aspartic acid (D)-amino acid entity; f) a serine (S)-amino acid entity; g) a cysteine (C)-amino acid entity; and h) a histidine (H)-amino acid entity; provided that: (i) at least one amino acid entity is not provided as a peptide of more than 20 amino acid residues in length; (ii) the amino acid entities of (a)-(h) are selected from Table 2; and (iii) the composition comprises fewer than 20 different amino acid entities.

In another aspect, the disclosure is directed to a composition comprising: a) a leucine (L)-amino acid entity; b) an isoleucine (I)-amino acid entity; c) a valine (V)-amino acid entity; d) a methionine (M)-amino acid entity; e) a cysteine (C)-amino acid entity; f) an arginine (R)-amino acid entity; g) a histidine (H)-amino acid entity; h) a tyrosine (Y)-amino acid entity; and i) a tryptophan (W)-amino acid entity; provided that: (i) at least one amino acid entity is not provided as a peptide of more than 20 amino acid residues in length; (ii) the amino acid entities of (a)-(i) are selected from Table 2; and (iii) the composition comprises fewer than 20 different amino acid entities.

In another aspect, the disclosure is directed to a composition comprising: a) a leucine (L)-amino acid entity; b) an isoleucine (I)-amino acid entity; c) a valine (V)-amino acid entity; d) a glutamic acid (E)-amino acid entity; e) an aspartic acid (D)-amino acid entity; f) a serine (S)-amino acid entity; g) a cysteine (C)-amino acid entity; h) an ornithine (Orn)-amino acid entity; and i) a histidine (H)-amino acid entity; provided that: (i) at least one amino acid entity is not provided as a peptide of more than 20 amino acid residues in length; (ii) the amino acid entities of (a)-(i) are selected from Table 2; and (iii) the composition comprises fewer than 20 different amino acid entities.

In another aspect, the disclosure is directed to a composition comprising: a) a leucine (L)-amino acid entity; b) an isoleucine (I)-amino acid entity; c) a valine (V)-amino acid entity; d) an aspartic acid (D)-amino acid entity; e) a cysteine (C)-amino acid entity; and f) an ornithine (Orn)-amino acid entity; provided that: (i) at least one amino acid entity is not provided as a peptide of more than 20 amino acid residues in length; (ii) the amino acid entities of (a)-(f) are selected from Table 2; and (iii) the composition comprises fewer than 20 different amino acid entities.

In another aspect, the disclosure is directed to a composition comprising: a) a leucine (L)-amino acid entity; b) an isoleucine (I)-amino acid entity; c) a valine (V)-amino acid entity; d) a glutamic acid (E)-amino acid entity; e) an aspartic acid (D)-amino acid entity; f) a serine (S)-amino acid entity; g) a cysteine (C)-amino acid entity; h) an ornithine (Orn)-amino acid entity; i) a histidine (H)-amino acid entity; j) a tyrosine (Y)-amino acid entity; and k) a tryptophan (W)-amino acid entity; provided that: (i) at least one amino acid entity is not provided as a peptide of more than 20 amino acid residues in length; (ii) the amino acid entities of (a)-(k) are selected from Table 2; and (iii) the composition comprises fewer than 20 different amino acid entities.

In another aspect, the disclosure is directed to a composition comprising: a) a leucine (L)-amino acid entity; b) an isoleucine (I)-amino acid entity; c) a valine (V)-amino acid entity; d) a glutamine (Q)-amino acid entity; e) a glycine (G)-amino acid entity; f) a serine (S)-amino acid entity; g) a methionine (M)-amino acid entity; h) a cysteine (C)-amino acid entity; i) an arginine (R)-amino acid entity; j) a histidine (H)-amino acid entity; k) a tyrosine (Y)-amino acid entity; and l) a tryptophan (W)-amino acid entity; provided that: (i) at least one amino acid entity is not provided as a peptide of more than 20 amino acid residues in length; (ii) the amino acid entities of (a)-(l) are selected from Table 2; and (iii) the composition comprises fewer than 20 different amino acid entities.

In another aspect, the disclosure is directed to a composition comprising: a) a glutamine (Q)-amino acid entity; b) a glutamic acid (E)-amino acid entity; c) an aspartic acid (D)-amino acid entity; d) a glycine (G)-amino acid entity; and e) a serine (S)-amino acid entity; provided that: (i) at least one amino acid entity is not provided as a peptide of more than 20 amino acid residues in length; (ii) the amino acid entities of (a)-(e) are selected from Table 2; and (iii) the composition comprises fewer than 20 different amino acid entities.

In another aspect, the disclosure is directed to a composition comprising: a) a leucine (L)-amino acid entity; b) an isoleucine (I)-amino acid entity; c) a valine (V)-amino acid entity; d) an ornithine (Orn)-amino acid entity; and e) an arginine (R)-amino acid entity; provided that: (i) at least one amino acid entity is not provided as a peptide of more than 20 amino acid residues in length; (ii) the amino acid entities of (a)-(e) are selected from Table 2; and (iii) the composition comprises fewer than 20 different amino acid entities.

In another aspect, the disclosure is directed to a composition comprising: a) a leucine (L)-amino acid entity; b) an isoleucine (I)-amino acid entity; c) a valine (V)-amino acid entity; d) a glutamic acid (E)-amino acid entity; e) an aspartic acid (D)-amino acid entity; f) a serine (S)-amino acid entity; g) an ornithine (Orn)-amino acid entity; and h) a histidine (H)-amino acid entity; provided that: (i) at least one amino acid entity is not provided as a peptide of more than 20 amino acid residues in length; (ii) the amino acid entities of (a)-(h) are selected from Table 2; and (iii) the composition comprises fewer than 20 different amino acid entities.

In another aspect, the disclosure is directed to a composition comprising: a) a leucine (L)-amino acid entity; b) an isoleucine (I)-amino acid entity; c) a valine (V)-amino acid entity; d) a methionine (M)-amino acid entity; and e) a cysteine (C)-amino acid entity; provided that: (i) at least one amino acid entity is not provided as a peptide of more than 20 amino acid residues in length; (ii) the amino acid entities of (a)-(e) are selected from Table 2; and (iii) the composition comprises fewer than 20 different amino acid entities.

In another aspect, the disclosure is directed to a composition comprising: a) a leucine (L)-amino acid entity; b) an isoleucine (I)-amino acid entity; c) a valine (V)-amino acid entity; d) a glutamine (Q)-amino acid entity; e) a glutamic acid (E)-amino acid entity; f) an aspartic acid (D)-amino acid entity; g) a glycine (G)-amino acid entity; h) a serine (S)-amino acid entity; i) a cysteine (C)-amino acid entity; and j) an ornithine (Orn)-amino acid entity; provided that: (i) at least one amino acid entity is not provided as a peptide of more than 20 amino acid residues in length; (ii) the amino acid entities of (a)-(j) are selected from Table 2; and (iii) the composition comprises fewer than 20 different amino acid entities.

In another aspect, the disclosure is directed to a composition comprising: a) a glutamine (Q)-amino acid entity; b) a glutamic acid (E)-amino acid entity; c) an aspartic acid (D)-amino acid entity; d) a glycine (G)-amino acid entity; e) an ornithine (Orn)-amino acid entity; and f) an arginine (R)-amino acid entity; provided that: (i) at least one amino acid entity is not provided as a peptide of more than 20 amino acid residues in length; (ii) the amino acid entities of (a)-(f) are selected from Table 2; and (iii) the composition comprises fewer than 20 different amino acid entities.

In another aspect, the disclosure is directed to a composition comprising: a) a leucine (L)-amino acid entity; b) an isoleucine (I)-amino acid entity; c) a valine (V)-amino acid entity; d) a cysteine (C)-amino acid entity; and e) a histidine (H)-amino acid entity; provided that: (i) at least one amino acid entity is not provided as a peptide of more than 20 amino acid residues in length; (ii) the amino acid entities of (a)-(e) are selected from Table 2; and (iii) the composition comprises fewer than 20 different amino acid entities.

In another aspect, the disclosure is directed to a composition comprising: a) a leucine (L)-amino acid entity; b) an isoleucine (I)-amino acid entity; c) a valine (V)-amino acid entity; d) a glutamic acid (E)-amino acid entity; e) an aspartic acid (D)-amino acid entity; and f) a glycine (G)-amino acid entity; provided that: (i) at least one amino acid entity is not provided as a peptide of more than 20 amino acid residues in length; (ii) the amino acid entities of (a)-(f) are selected from Table 2; and (iii) the composition comprises fewer than 20 different amino acid entities.

In another aspect, the disclosure is directed to a composition comprising: a) a leucine (L)-amino acid entity; b) an isoleucine (I)-amino acid entity; c) a valine (V)-amino acid entity; d) a cysteine (C)-amino acid entity or a NAC entity; and e) an ornithine (Orn)-amino acid entity; provided that: (i) at least one amino acid entity is not provided as a peptide of more than 20 amino acid residues in length; (ii) the amino acid entities of (a)-(e) are selected from Table 2; and (iii) the composition comprises fewer than 20 different amino acid entities.

In another aspect, the disclosure is directed to a composition comprising: a) a glutamic acid (E)-amino acid entity; b) an aspartic acid (D)-amino acid entity; and c) a glycine (G)-amino acid entity; provided that: (i) at least one amino acid entity is not provided as a peptide of more than 20 amino acid residues in length; (ii) the amino acid entities of (a)-(c) are selected from Table 2; and (iii) the composition comprises fewer than 20 different amino acid entities.

In another aspect, the disclosure is directed to a composition comprising: a) a glutamine (Q)-amino acid entity; and b) a glutamic acid (E)-amino acid entity; provided that: (i) at least one amino acid entity is not provided as a peptide of more than 20 amino acid residues in length; (ii) the amino acid entities of (a)-(b) are selected from Table 2; and (iii) the composition comprises fewer than 20 different amino acid entities.

In another aspect, the disclosure is directed to a composition comprising: a) a methionine (M)-amino acid entity; and b) a cysteine (C)-amino acid entity; provided that: (i) at least one amino acid entity is not provided as a peptide of more than 20 amino acid residues in length; (ii) the amino acid entities of (a)-(b) are selected from Table 2; and (iii) the composition comprises fewer than 20 different amino acid entities.

In another aspect, the disclosure is directed to a composition comprising: a) an ornithine (Orn)-amino acid entity; and b) an arginine (R)-amino acid entity; provided that: (i) at least one amino acid entity is not provided as a peptide of more than 20 amino acid residues in length; (ii) the amino acid entities of (a)-(b) are selected from Table 2; and (iii) the composition comprises fewer than 20 different amino acid entities.

In another aspect, the disclosure is directed to a composition comprising: a) a leucine (L)-amino acid entity; b) an isoleucine (I)-amino acid entity; c) a valine (V)-amino acid entity; and d) an aspartate (D)-amino acid entity; provided that: (i) at least one amino acid entity is not provided as a peptide of more than 20 amino acid residues in length; (ii) the amino acid entities of (a)-(d) are selected from Table 2; and (iii) the composition comprises fewer than 20 different amino acid entities.

In another aspect, the disclosure is directed to a pharmaceutical composition comprising: a) a composition described herein; and b) one or more pharmaceutically acceptable excipients; provided that: i) at least one amino acid entity is not provided as a peptide of more than 20 amino acid residues in length; and ii) the amino acid entities in the composition are selected from Table 2.

In another aspect, the disclosure is directed to a method for treating one or more symptoms selected from immobilization, malnutrition, fasting, aging, autophagy, reduced protein synthesis, anabolic resistance, junction integrity, insulin resistance, decreased mitochondrial biogenesis, decreased myogenesis or myotube growth, anaplerosis, or an energy deficit, wherein the method comprises administering to a subject in need thereof an effective amount of a composition described herein; provided that: i) at least one amino acid entity is not provided as a peptide of more than 20 amino acid residues in length; and ii) the amino acid entities in the composition are selected from Table 2.

In another aspect, the disclosure is directed to a method for improving muscle function, wherein the method comprises administering to a subject in need thereof an effective amount of the composition described herein; provided that: i) at least one amino acid entity is not provided as a peptide of more than 20 amino acid residues in length; and ii) the amino acid entities in the composition are selected from Table 2.

In another aspect, the disclosure is directed to a dietary composition comprising a composition described herein provided that: i) at least one amino acid entity is not provided as a peptide of more than 20 amino acid residues in length; and (ii) the amino acid entities in the composition are selected from Table 2.

In another aspect, the disclosure is directed to a method of providing amino acid entities to a subject comprising administering to the subject an effective amount of a as described herein; provided that: i) at least one amino acid entity is not provided as a peptide of more than 20 amino acid residues in length; and (ii) the amino acid entities in the composition are selected from Table 2.

In another aspect, the disclosure is directed to a method of manufacturing or making a composition as described herein.

In another aspect, the disclosure is directed to a method for increasing myogenesis, wherein the method comprises administering to a subject in need thereof an effective amount of a composition described herein; provided that: i) at least one amino acid entity is not provided as a peptide of more than 20 amino acid residues in length; and ii) the amino acid entities in the composition are selected from Table 2.

In another aspect, the disclosure is directed to a method for increasing muscle protein synthesis, wherein the method comprises administering to a subject in need thereof an effective amount of a composition described herein; provided that: i) at least one amino acid entity is not provided as a peptide of more than 20 amino acid residues in length; and ii) the amino acid entities in the composition are selected from Table 2.

In another aspect, the disclosure is directed to a method for increasing muscle mass, wherein the method comprises administering to a subject in need thereof an effective amount of a composition described herein; provided that: i) at least one amino acid entity is not provided as a peptide of more than 20 amino acid residues in length; and ii) the amino acid entities in the composition are selected from Table 2.

In another aspect, the disclosure is directed to a method for improving muscle quality, wherein the method comprises administering to a subject in need thereof an effective amount of a composition described herein; provided that: i) at least one amino acid entity is not provided as a peptide of more than 20 amino acid residues in length; and ii) the amino acid entities in the composition are selected from Table 2.

### DETAILED DESCRIPTION

The present invention provides, at least in part, methods and compositions comprising at least five different amino acid entities. In some embodiments, the composition is capable of one, two, three, or all of:

a) activating mTORC1;

b) activating protein synthesis and/or inhibiting protein breakdown;

c) improving, e.g., increasing, insulin sensitivity

d) reducing inflammation;

e) improving myogenesis or myotube growth;

f) improving muscle mass;

g) improving muscle function;

h) increasing muscle growth; or

i) lowering muscle fat.

In some embodiments, at least one amino acid entity in the compositions is not provided as a peptide of more than 20 amino acid residues in length.

In some embodiments, the composition comprises at least five different amino acid entities selected from the group consisting of a leucine (L)-amino acid entity, an isoleucine (I)-amino acid entity, a valine (V)-amino acid entity, a glutamine (Q)-amino acid entity, a glutamic acid (E)-amino acid entity, an aspartic acid (D)-amino acid entity, a glycine (G)-amino acid entity, a serine (S)-amino acid entity, a methionine (M)-amino acid entity, a cysteine (C)-amino acid entity or NAC entity, an ornithine (Orn)-amino acid entity, an arginine (R)-amino acid entity, a histidine (H)-amino acid entity, a tyrosine (Y)-amino acid entity, and a tryptophan (W)-amino acid entity. In some embodiments, at least one amino acid entity is not a peptide of more than 20 amino acid residues in length.

In some embodiments, the composition is capable of improving one or more physiological symptoms selected from one, two, three, four, five, six, seven, eight, nine, ten, or more (e.g., all) of immobilization, malnutrition, fasting, aging, autophagy, reduced protein synthesis, anabolic resistance, neuromuscular junction integrity, insulin resistance, decreased mitochondrial biogenesis, anaplerosis, myogenesis, or an energy deficit.

The composition can be administered to a subject to provide a beneficial effect in one or both of improving muscle function or treating (e.g., reversing, reducing, ameliorating, or preventing) a muscle disease or disorder. In some embodiments, the composition can be administered to treat (e.g., reverse, reduce, ameliorate, or prevent) a subject having or identified as having decreased muscle function due to aging, injury, atrophy, infection, or disease. In some embodiments, administration of the composition results in an improvement in one, two, or more of strength, stamina, or endurance in a subject, e.g., in a human. In some embodiments, administration of the composition results in an improvement, e.g., an increase, in one, two, or more of muscle cross sectional area, fiber quality, and lean muscle mass in a subject, e.g., in a human.

In some embodiments, the subject has a rare muscle disease. In some embodiments, the subject has sarcopenia, muscle deterioration, decay, atrophy, cachexia, steroid myopathy, muscular dystrophy, or myopenia. In some embodiments, the subject has a fracture or other trauma. In some embodiments, the subject has a drug-induced myopathy. In some embodiments, the subject has a statin-induced myopathy. In some embodiments, the subject has a steroid-induced myopathy. In some embodiments, the subject has an immunosuppressant-induced myopathy. In some embodiments, the subject has a chemotherapeutic-induced myopathy. In some embodiments, the subject has an alcohol-induced myopathy.

In some embodiments, the subject exhibits muscle loss related to one or both of immobilization or muscle disuse following injury. In some embodiments, the subject has, or is recovering from, a surgery, e.g., rotator cuff surgery, knee surgery, or hip surgery, or has worn a cast prior to administration of the composition. In some embodiments, the subject has had, or is recovering from, a hip fracture-related myopenia prior to administration of the composition. In some embodiments, the subject has had, or is recovering from, a joint replacement prior to administration of the composition. In some embodiments, the subject has had, or is recovering from, an injury repair surgery.

In some embodiments, the subject has, or is recovering from, ventilator-induced diaphragmatic dystrophy or ventilator-induced diaphragmatic dysfunction prior to administration of the composition. In some embodiments, the subject has had one or both of ICU-acquired or burns-related myopathies.

In some embodiments, the subject has disease-related cachexia, e.g., a disease-related cachexia selected from chronic obstructive pulmonary disease (COPD), congestive heart failure (CHF), chronic kidney disease (CKD), and cancer prior to administration of the composition.

In some embodiments, the subject has perceived muscle weakness, e.g., chronic fatigue syndrome. In some embodiments, the subject has a cancer-associated muscle weakness. In some embodiments, the subject has a neuromuscular disorder, e.g., myasthenia gravis or Lambert-Eaton myasthenic syndrome. In some embodiments, the subject has muscular dystrophy, e.g., Duchenne muscular dystrophy, Becker muscular dystrophy, facioscapulohumeral muscular dystrophy, or myotonic dystrophy. In some embodiments, the subject has inflammatory myopathy, e.g., polymyositis or dermatomyositis.

In some embodiments, the subject has one, two, or more (e.g., all) of low sodium levels (e.g., hyponatremia), low potassium levels (e.g., hypokalemia), or a calcium deficiency or relatively high calcium levels (e.g., hypercalcemia).

In some embodiments, the subject has muscle weakness associated with nerve damage, e.g., neuralgia or peripheral neuropathy. In some embodiments, the subject has a bone weakness disease, e.g., osteomalacia, osteogenesis imperfecta, rickets, or hypophosphatasia.

In some embodiments, the subject has experienced a stroke or a transient ischemic attack. In some embodiments, the subject has an autoimmune disease, e.g., Graves' disease.

In some embodiments, the subject has hypothyroidism. In some embodiments, the subject has amyotrophic lateral sclerosis (ALS).

Also provided is a method of treating one, two, three, four, five, six, seven, eight, nine, or more (e.g., all) of immobilization, malnutrition, fasting, aging, autophagy, reduced protein synthesis, anabolic resistance, junction integrity (e.g., neuromuscular junction integrity), insulin resistance, decreased mitochondrial biogenesis, an energy deficit, or anaplerosis in a subject that includes administering to a subject in need thereof an effective amount of a pharmaceutical composition including defined amino acid components. In some embodiments, the subject has a rare muscle disease. In some embodiments, the subject has sarcopenia, muscle deterioration, decay, atrophy, cachexia, drug-induced myopathy, muscular dystrophy, or myopenia. In some embodiments, the subject has a fracture or other trauma. In some embodiments, the subject has a drug-induced myopathy. In some embodiments, the subject has a statin-induced myopathy. In some embodiments, the subject has a steroid-induced myopathy. In some embodiments, the subject has an immunosuppressant-induced myopathy. In some embodiments, the subject has a chemotherapeutic-induced myopathy. In some embodiments, the subject has an alcohol-induced myopathy.

The subject may exhibit an improvement in muscle function after administration of a composition comprising described herein. For example the composition may be administered to the subject for a treatment period of, e.g., two weeks, three weeks, four weeks, five weeks, six weeks, seven weeks, eight weeks, nine weeks, 10 weeks, 11 weeks, 12 weeks, 13 weeks, 14 weeks, 15 weeks, 16 weeks, or longer at a dose of, e.g., about 4 total grams per day to about 80 total grams per day (e.g., a total of about 18 g per day, 48 g per day, 68 g per day or a total of about 72 g per day).

Treatment with the composition can result in improved muscle function in a subject, e.g., by one, two, three, four, five or more (e.g., all) of activating mTORC1; improving insulin sensitivity; activating muscle protein synthesis; scavenging reactive oxygen species (ROS); decreasing inflammation (e.g., muscle inflammation); inhibiting catabolism; detoxifying ammonia; or decreasing fibrosis progression.

Improvements in muscle function can be assessed by performing metrics selected from one, two, three, four, or all of a maximal isometric knee strength test (e.g., to determine changes in muscle strength), magnetic resonance imaging (MRI, e.g., to determine total muscle volume, e.g., thigh muscle volume), muscle biopsy (e.g., to determine muscle fiber quality), a dual-energy x-ray absorptiometry (DEXA) scan (e.g., to determine body composition including lean mass and fat-free mass), and electrical impedance myography (EIM) (e.g., to determine muscle health, such as resistive and capacitive properties of muscle tissue and sensitivity to disuse-related atrophy).

In some embodiments, the composition is for use as a medicament in improving muscle function in a subject. In some embodiments, the composition is for use as a medicament in treating a muscle disease or disorder in a subject.

In some embodiments, the composition is for use in the manufacture of a medicament for improving muscle function in a subject. In some embodiments, the composition including amino acid entities is for use in the manufacture of a medicament for treating a muscle disease or disorder in a subject.

Additionally, the composition is useful as a dietary supplement.

One embodiment provides a nutritional supplement, dietary formulation, functional food, medical food, food, or beverage comprising a composition described herein. Another embodiment provides a nutritional supplement, dietary formulation, functional food, medical food, food, or beverage comprising a composition described herein for use in the management of any of the diseases or disorders described herein.

One embodiment provides a method of maintaining or improving muscle health, muscle function, muscle functional performance, or muscle strength, comprising administering to a subject an effective amount of a composition described herein. Another embodiment provides a method of providing nutritional support or supplementation to a subject suffering from muscle atrophy comprising administering to the subject an effective amount of a composition described herein. Yet another embodiment provides a method of providing nutritional support or supplementation that aids in the management of muscle atrophy to a subject comprising administering to the subject in need thereof an effective amount of a composition described herein.

### Definitions

Terms used in the claims and specification are defined as set forth below unless otherwise specified.

It must be noted that, as used in the specification and the appended claims, the singular forms “a,” “an” and “the” include plural referents unless the context clearly dictates otherwise.

As used herein, the term “amino acid entity” refers to an amino acid in one or both of free form or salt form, an amino acid residue of a peptide (e.g., of a dipeptide, oligopeptide, or polypeptide), a derivative of an amino acid, a precursor of an amino acid, or a metabolite of an amino acid.

As used herein the term “XXX amino acid entity” refers to an amino acid entity that if a free amino acid, comprises free XXX or XXX in salt form; if a peptide, refers to a peptide comprising an XXX residue; if a derivative, refers to a derivative of XXX; if a precursor, refers to a precursor of XXX; and if a metabolite, refers to a XXX metabolite. For example, where XXX is leucine (L), then L-amino acid entity refers to free L or L in salt form, a peptide comprising a L residue, a L derivative, a L precursor, or a metabolite of L; where XXX is isoleucine (I), then I-amino acid entity refers to free I or I in salt form, a peptide comprising an I residue, an I derivative, an I precursor, or a metabolite of I; where XXX is valine (V), then V-amino acid entity refers to free V or V in salt form, a peptide comprising a V residue, a V derivative, a V precursor, or a metabolite of V; where XXX is glutamine (Q), then Q-amino acid entity refers to free Q or Q in salt form, a peptide comprising a Q residue, a Q derivative, a Q precursor, or a metabolite of Q; where XXX is glutamic acid (E), then E-amino acid entity refers to free E or E in salt form, a peptide comprising a E residue, an E derivative, a E precursor, or a metabolite of E; where XXX is aspartic acid (D), then D-amino acid entity refers to free D or D in salt form, a peptide comprising a D residue, a D derivative, a D precursor, or a metabolite of D; where XXX is glycine (G), then G-amino acid entity refers to free G or G in salt form, a peptide comprising a G residue, a G derivative, a G precursor, or a metabolite of G; where XXX is serine (S), then S-amino acid entity refers to free S or S in salt form, a peptide comprising a S residue, a S derivative, a S precursor, or a metabolite of S; where XXX is methionine (M), then M-amino acid entity refers to free M or M in salt form, a peptide comprising a M residue, a M derivative, a M precursor, or a metabolite of M; where XXX is cysteine (C), then C-amino acid entity refers to free C or C in salt form, a peptide comprising a C residue, a C derivative, a C precursor, or a metabolite of C; where XXX is ornithine (Orn), then Orn-amino acid entity refers to free Orn or Orn in salt form, a peptide comprising an Orn residue, an Orn derivative, an Orn precursor, or a metabolite of Orn; where XXX is arginine (R), then R-amino acid entity refers to free R or R in salt form, a peptide comprising a R residue, a R derivative, a R precursor, or a metabolite of R; where XXX is histidine (H), then H-amino acid entity refers to free H or H in salt form, a peptide comprising a H residue, a H derivative, a H precursor, or a metabolite of H; where XXX is tyrosine (Y), then Y-amino acid entity refers to free Y or Y in salt form, a peptide comprising a Y residue, a Y derivative, a Y precursor, or a metabolite of Y; and where XXX is tryptophan (W), then W-amino acid entity refers to free W or W in salt form, a peptide comprising a W residue, a W derivative, a W precursor, or a metabolite of W.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

An “amino acid” refers to an organic compound having an amino group (—NH₂), a carboxylic acid group (—C(═O)OH), and a side chain bonded through a central carbon atom, and includes essential and non-amino acids, as well as natural and unnatural amino acids.

The proteogenic amino acids, shown below, are known by three- and one-letter abbreviations in addition to their full names. For a given amino acid, these abbreviations are used interchangeably herein. For example, Leu, L or leucine all refer to the amino acid leucine; Ile, I or isoleucine all refer to the amino acid isoleucine; Val, V or valine all refer to the amino acid valine; Arg, R or arginine all refer to the amino acid arginine; and Gln, Q or glutamine all refer to the amino acid glutamine. Likewise, the non-natural amino acid derivative N-acetylcysteine may be referred to interchangeably by “NAC” or “N-acetylcysteine.” Amino acids may be present as D- or L-isomers. Unless otherwise indicated, amino acids referred to herein are L-isomers of amino acids.

TABLE 1
Amino acid names and abbreviations

| Amino acid | Three-letter | One-letter |
|---|---|---|
| Alanine | Ala | A |
| Arginine | Arg | R |
| Asparagine | Asn | N |
| Aspartic | Asp | D |
| acid/Aspartate | | |
| Cysteine | Cys | C |
| Glutamic | Glu | E |
| acid/Glutamate | | |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

A “branched chain amino acid” is an amino acid selected from leucine, isoleucine, and valine.

The term “effective amount” as used herein means an amount of an amino acid, or pharmaceutical composition which is sufficient enough to significantly and positively modify the symptoms and/or conditions to be treated (e.g., provide a positive clinical response). The effective amount of an active ingredient for use in a pharmaceutical composition will vary with the particular condition being treated, the severity of the condition, the duration of treatment, the nature of concurrent therapy, the particular active ingredient(s) being employed, the particular pharmaceutically-acceptable excipient(s) and/or carrier(s) utilized, and like factors with the knowledge and expertise of the attending physician.

A “pharmaceutical composition” described herein comprises at least one amino acid and a pharmaceutically acceptable carrier or excipient. In some embodiments, the pharmaceutical composition is used as a therapeutic, a nutraceutical, a medical food, or as a supplement.

The term “pharmaceutically acceptable” as used herein, refers to amino acids, materials, excipients, compositions and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

A composition, formulation or product is “therapeutic” if it provides a beneficial clinical effect. A beneficial clinical effect can be shown by lessening the progression of a disease and/or alleviating one or more symptoms of the disease.

A “unit dose” or “unit dosage” as used herein means an amount or dose of medicine prepared in an individual packet or container for convenience, safety, or monitoring. A “unit dose” or “unit dosage” comprises the drug product or drug products in the form in which they are marketed for use, with a specific mixture of active ingredients and inactive components (excipients), in a particular configuration (such as a capsule shell, for example), and apportioned into a particular dose.

As used herein, the terms “treat,” “treating,” or “treatment” refer in one embodiment, to ameliorating, e.g., decreased muscle function (e.g., relative to a health subject), a muscle disease, or a muscle disorder (i.e., slowing or arresting or reducing the development of the disease or disorder or at least one of the clinical symptoms thereof). In another embodiment, “treat,” “treating,” or “treatment” refers to alleviating or ameliorating at least one physical parameter including those which may not be discernible by the patient. In yet another embodiment, “treat,” “treating,” or “treatment” refers to modulating a symptom of decreased muscle function (e.g., relative to a health subject), a muscle disease, or a muscle disorder, either physically, (e.g., stabilization of a discernible symptom), physiologically, (e.g., stabilization of a physical parameter), or both. In yet another embodiment, “treat,” “treating,” or “treatment” refers to preventing or delaying the onset or development or progression of decreased muscle function (e.g., relative to a health subject), a muscle disease, or a muscle disorder.

### Determination of Amino Acid Weight Percent and Amino Acid Ratios in a Composition

The weight ratio of a particular amino acid or particular amino acids in a composition or mixture of amino acids is the ratio of the weight of the particular amino acid or amino acids in the composition or mixture compared to the total weight of amino acids present in the composition or mixture. This value is calculated by dividing the weight of the particular amino acid or of the particular amino acids in the composition or mixture by the weight of all amino acids present in the composition or mixture.

### Compositions Comprising Amino Acid Entities

The present disclosure provides compositions, e.g., pharmaceutical compositions, comprising amino acid entities. These pharmaceutical compositions are made up of amino acid entities including amino acids in one or both of free form or salt form, amino acid residues of a peptide (e.g., of a dipeptide, oligopeptide, or polypeptide), derivatives of an amino acid, precursors of an amino acid, or metabolites of an amino acid. For example, the compositions can include a leucine (L)-amino acid entity, an isoleucine (I)-amino acid entity, a valine (V)-amino acid entity, a histidine (H)-amino acid entity; and a N-acetylcysteine (NAC) entity or a cysteine (C)-amino acid entity, e.g., NAC (Table 2). In particular, at least one amino acid entity is not a peptide of more than 20 amino acid residues in length.

TABLE 2
Amino acid entities include amino acids, precursors, metabolites,
and derivatives of the compositions described herein.

| | Ex- | | | | |
|---|---|---|---|---|---|
| | emplary | | | | |
| | Amino | | | Deriv- | |
| | Acid | Precursors | Metabolites | atives | Salts |
| L | L-Leucine | Oxo- | HMB (beta- | D-Leucine; | |
| | | leucine | hydroxy- | N-Acetyl- | |
| | | | beta-methy- | Leucine | |
| | | | butyrate); | | |
| | | | Oxo-leucine; | | |
| | | | Isovaleryl- | | |
| | | | CoA | | |
| I | L- | 2-Oxo- | 2-Oxo-3- | D- | |
| | Isoleucine | 3-methyl- | methyl- | Isoleucine; | |
| | | valerate; | valerate; | N-Acetyl- | |
| | | Threonine | Methyl- | Isoleucine | |
| | | | butyrl- | | |
| | | | CoA | | |
| V | L-Valine | 2-Oxo- | Isobutryl- | N-Acetyl- | |
| | | valerate | CoA; | Valine | |
| E | L- | 2-Oxo- | Glutathione; | | |
| | Glutamate | glutarate | Glutamine; | | |
| | | | Carbamoyl-P; | | |
| D | L- | Fumarate | Adenylo- | | |
| | Aspartate | | succinate | | |
| G | Glycine | L-Serine | Glutathione; | | |
| | | | L-Serine | | |
| M | L- | Homo- | | | |
| | Methionine | cysteine; | | | |
| | | L-Cysteine; | | | |
| C | L- | Serine; | Glutathione; | N- | |
| | Cysteine | Acetyl- | Cysta- | acetyl- | |
| | | serine; | thionine; | cysteine | |
| | | Cysta- | Homo- | (Nac); | |
| | | thionine; | cysteine; | Cystine; | |
| | | | Methionine | Cysteamine | |
| S | L-Serine | Phospho- | Glycine, | | |
| | | serine, | Tryptophan, | | |
| | | P-hydroxy- | Acetylserine, | | |
| | | pyruvate, | Cysta- | | |
| | | Glycine | thionine, | | |
| | | | Phosphati- | | |
| | | | dylserine | | |
| R | L- | Arginino | Ornithine; | D-Arginine; | |
| | Arginine | succinate; | Citrulline; | N-Acetyl- | |
| | | Citrulline; | Agmatine; | Arginine; | |
| | | Aspartate; | Creatine | | |
| | | Glutamate | | | |
| Orni- | L- | L-Arginine, | Citrulline | | Ornithine |
| thine | Ornithine | Glycine | | | α-keto- |
| | | | | | glutarate, |
| | | | | | Ornithine |
| | | | | | HCl |
| NAC | N- | Serine; | Glutathione; | D-Cysteine; | |
| | Acetyl- | Acetyl- | Cysta- | L-Cysteine; | |
| | cysteine | serine; | thionine; | Cystine; | |
| | | Cysta- | Homo- | Cysteamine | |
| | | | cysteine; | | |
| | | thionine; | Methionine | | |
| H | L- | Histidinol; | Carnosine; | D- | |
| | Histidine | Histidinal; | Histamine; | Histidine; | |
| | | Ribose-5- | Urocanate | N-Acetyl- | |
| | | phosphate | | Histidine | |
| Y | L- | L-Phenyl- | | | |
| | Tyrosine | alanine; | | | |
| W | L- | L-Serine | Kynurenine; | | |
| | Tryptophan | | Serotonin | | |
| Q | L- | Glutamate | Carbamoyl- | D- | |
| | Glutamine | | P; | Glutamine; | |
| | | | Glutamate | N-Acetyl- | |
| | | | | Glutamine; | |

In certain embodiments, the composition comprises: a) a leucine (L)-amino acid entity, b) an isoleucine (I)-amino acid entity, c) a valine (V)-amino acid entity, d) an aspartic acid (D)-amino acid entity, e) a methionine (M)-amino acid entity, f) a cysteine (C)-amino acid entity, g) an arginine (R)-amino acid entity, h) a histidine (H)-amino acid entity, i) a tyrosine (Y)-amino acid entity, and j) a tryptophan (W)-amino acid entity, provided that: i) at least one amino acid entity is not provided as a peptide of more than 20 amino acid residues in length, and (ii) the amino acid entities of (a)-(j) are selected from Table 2. The total wt. % of (a)-(j) may be greater than the total wt. % of other amino acid entities in the composition.

In certain embodiments, the composition comprises: a) a leucine (L)-amino acid entity, b) an isoleucine (I)-amino acid entity, c) a valine (V)-amino acid entity, d) a glutamine (Q)-amino acid entity, e) a glutamic acid (E)-amino acid entity, f) an aspartic acid (D)-amino acid entity, g) a glycine (G)-amino acid entity, h) a serine (S)-amino acid entity, i) a methionine (M)-amino acid entity, j) a cysteine (C)-amino acid entity, k) an ornithine (Orn)-amino acid entity, l) an arginine (R)-amino acid entity, m) a histidine (H)-amino acid entity, n) a tyrosine (Y)-amino acid entity, and o) a tryptophan (W)-amino acid entity, provided that: i) at least one amino acid entity is not provided as a peptide of more than 20 amino acid residues in length, and (ii) the amino acid entities of (a)-(o) are selected from Table 2. The total wt. % of (a)-(o) may be greater than the total wt. % of other amino acid entities in the composition.

In certain embodiments, the composition comprises: a) a leucine (L)-amino acid entity, b) an isoleucine (I)-amino acid entity, c) a valine (V)-amino acid entity, d) a glutamic acid (E)-amino acid entity, e) an aspartic acid (D)-amino acid entity, f) a serine (S)-amino acid entity, g) a cysteine (C)-amino acid entity, and h) a histidine (H)-amino acid entity, provided that: i) at least one amino acid entity is not provided as a peptide of more than 20 amino acid residues in length, and ii) the amino acid entities of (a)-(h) are selected from Table 2. The total wt. % of (a)-(h) may be greater than the total wt. % of other amino acid entities in the composition.

In certain embodiments, the composition comprises: a) a leucine (L)-amino acid entity, b) an isoleucine (I)-amino acid entity, c) a valine (V)-amino acid entity, d) a methionine (M)-amino acid entity, e) a cysteine (C)-amino acid entity, f) an arginine (R)-amino acid entity, g) a histidine (H)-amino acid entity, h) a tyrosine (Y)-amino acid entity, and i) a tryptophan (W)-amino acid entity, provided that: i) at least one amino acid entity is not provided as a peptide of more than 20 amino acid residues in length, and ii) the amino acid entities of (a)-(i) are selected from Table 2. The total wt. % of (a)-(i) may be greater than the total wt. % of other amino acid entities in the composition.

In certain embodiments, the composition comprises: a) a leucine (L)-amino acid entity, b) an isoleucine (I)-amino acid entity, c) a valine (V)-amino acid entity, d) a glutamic acid (E)-amino acid entity, e) an aspartic acid (D)-amino acid entity, f) a serine (S)-amino acid entity, g) a cysteine (C)-amino acid entity, h) an ornithine (Orn)-amino acid entity, and i) a histidine (H)-amino acid entity, provided that: i) at least one amino acid entity is not provided as a peptide of more than 20 amino acid residues in length, and ii) the amino acid entities of (a)-(i) are selected from Table 2. The total wt. % of (a)-(i) may be greater than the total wt. % of other amino acid entities in the composition.

In certain embodiments, the composition comprises: a) a leucine (L)-amino acid entity, b) an isoleucine (I)-amino acid entity, c) a valine (V)-amino acid entity, d) an aspartic acid (D)-amino acid entity, e) a cysteine (C)-amino acid entity, and f) an ornithine (Orn)-amino acid entity, provided that: i) at least one amino acid entity is not provided as a peptide of more than 20 amino acid residues in length, and ii) the amino acid entities of (a)-(f) are selected from Table 2. The total wt. % of (a)-(f) may be greater than the total wt. % of other amino acid entities in the composition.

In certain embodiments, the composition comprises: a) a leucine (L)-amino acid entity, b) an isoleucine (I)-amino acid entity, c) a valine (V)-amino acid entity, d) a glutamic acid (E)-amino acid entity, e) an aspartic acid (D)-amino acid entity, f) a serine (S)-amino acid entity, g) a cysteine (C)-amino acid entity, h) an ornithine (Orn)-amino acid entity, i) a histidine (H)-amino acid entity, j) a tyrosine (Y)-amino acid entity, and k) a tryptophan (W)-amino acid entity, provided that: i) at least one amino acid entity is not provided as a peptide of more than 20 amino acid residues in length, and (ii) the amino acid entities of (a)-(k) are selected from Table 2. The total wt. % of (a)-(k) may be greater than the total wt. % of other amino acid entities in the composition.

In certain embodiments, the composition comprises: a) a leucine (L)-amino acid entity, b) an isoleucine (I)-amino acid entity, c) a valine (V)-amino acid entity, d) a glutamine (Q)-amino acid entity, e) a glycine (G)-amino acid entity, f) a serine (S)-amino acid entity, g) a methionine (M)-amino acid entity, h) a cysteine (C)-amino acid entity, i) an arginine (R)-amino acid entity, j) a histidine (H)-amino acid entity, k) a tyrosine (Y)-amino acid entity, and l) a tryptophan (W)-amino acid entity, provided that: i) at least one amino acid entity is not provided as a peptide of more than 20 amino acid residues in length, and (ii) the amino acid entities of (a)-(l) are selected from Table 2. The total wt. % of (a)-(l) may be greater than the total wt. % of other amino acid entities in the composition.

In certain embodiments, the composition comprises: a) a glutamine (Q)-amino acid entity, b) a glutamic acid (E)-amino acid entity, c) an aspartic acid (D)-amino acid entity, d) a glycine (G)-amino acid entity, and e) a serine (S)-amino acid entity, provided that: i) at least one amino acid entity is not provided as a peptide of more than 20 amino acid residues in length, and (ii) the amino acid entities of (a)-(e) are selected from Table 2. The total wt. % of (a)-(e) may be greater than the total wt. % of other amino acid entities in the composition.

In certain embodiments, the composition comprises: a) a leucine (L)-amino acid entity, b) an isoleucine (I)-amino acid entity, c) a valine (V)-amino acid entity, d) an ornithine (Orn)-amino acid entity, and e) an arginine (R)-amino acid entity, provided that: i) at least one amino acid entity is not provided as a peptide of more than 20 amino acid residues in length, and (ii) the amino acid entities of (a)-(e) are selected from Table 2. The total wt. % of (a)-(e) may be greater than the total wt. % of other amino acid entities in the composition.

In certain embodiments, the composition comprises: a) a leucine (L)-amino acid entity, b) an isoleucine (I)-amino acid entity, c) a valine (V)-amino acid entity, d) a glutamic acid (E)-amino acid entity, e) an aspartic acid (D)-amino acid entity, f) a serine (S)-amino acid entity, g) an ornithine (Orn)-amino acid entity, and h) a histidine (H)-amino acid entity, provided that: i) at least one amino acid entity is not provided as a peptide of more than 20 amino acid residues in length, and (ii) the amino acid entities of (a)-(h) are selected from Table 2. The total wt. % of (a)-(h) may be greater than the total wt. % of other amino acid entities in the composition.

In certain embodiments, the composition comprises: a) a leucine (L)-amino acid entity, b) an isoleucine (I)-amino acid entity, c) a valine (V)-amino acid entity, d) a methionine (M)-amino acid entity, and e) a cysteine (C)-amino acid entity, provided that: i) at least one amino acid entity is not provided as a peptide of more than 20 amino acid residues in length, and (ii) the amino acid entities of (a)-(e) are selected from Table 2. The total wt. % of (a)-(e) may be greater than the total wt. % of other amino acid entities in the composition.

In certain embodiments, the composition comprises: a) a leucine (L)-amino acid entity, b) an isoleucine (I)-amino acid entity, c) a valine (V)-amino acid entity, d) a glutamine (Q)-amino acid entity, e) a glutamic acid (E)-amino acid entity, f) an aspartic acid (D)-amino acid entity, g) a glycine (G)-amino acid entity, h) a serine (S)-amino acid entity, i) a cysteine (C)-amino acid entity, and j) an ornithine (Orn)-amino acid entity, provided that: i) at least one amino acid entity is not provided as a peptide of more than 20 amino acid residues in length, and (ii) the amino acid entities of (a)-(j) are selected from Table 2. The total wt. % of (a)-(j) may be greater than the total wt. % of other amino acid entities in the composition.

In certain embodiments, the composition comprises: a) a leucine (L)-amino acid entity, b) an isoleucine (I)-amino acid entity, c) a valine (V)-amino acid entity, d) a histidine (H)-amino acid entity, and e) a cysteine (C)-amino acid entity, provided that: i) at least one amino acid entity is not provided as a peptide of more than 20 amino acid residues in length, and (ii) the amino acid entities of (a)-(e) are selected from Table 2. The total wt. % of (a)-(e) may be greater than the total wt. % of other amino acid entities in the composition.

In certain embodiments, the composition comprises: a) a glutamine (Q)-amino acid entity, b) a glutamic acid (E)-amino acid entity, c) an aspartic acid (D)-amino acid entity, d) a glycine (G)-amino acid entity, e) an ornithine (Orn)-amino acid entity, and f) an arginine (R)-amino acid entity, provided that: i) at least one amino acid entity is not provided as a peptide of more than 20 amino acid residues in length, and (ii) the amino acid entities of (a)-(f) are selected from Table 2. The total wt. % of (a)-(f) may be greater than the total wt. % of other amino acid entities in the composition.

In certain embodiments, the composition comprises: a) a leucine (L)-amino acid entity, b) an isoleucine (I)-amino acid entity, c) a valine (V)-amino acid entity, d) a cysteine (C)-amino acid entity, and e) an ornithine (Orn)-amino acid entity, provided that: i) at least one amino acid entity is not provided as a peptide of more than 20 amino acid residues in length, and (ii) the amino acid entities of (a)-(e) are selected from Table 2. The total wt. % of (a)-(e) may be greater than the total wt. % of other amino acid entities in the composition.

In certain embodiments, the composition comprises: a) a glutamate (E)-amino acid entity, b) a aspartate (D)-amino acid entity, and c) a glycine (G)-amino acid entity, provided that: i) at least one amino acid entity is not provided as a peptide of more than 20 amino acid residues in length, and (ii) the amino acid entities of (a)-(c) are selected from Table 2. The total wt. % of (a)-(c) may be greater than the total wt. % of other amino acid entities in the composition.

In certain embodiments, the composition comprises: a) a leucine (L)-amino acid entity, b) an isoleucine (I)-amino acid entity, c) a valine (V)-amino acid entity, d) a glutamate (E)-amino acid entity, e) a aspartate (D)-amino acid entity, and f) a glycine (G)-amino acid entity, provided that: i) at least one amino acid entity is not provided as a peptide of more than 20 amino acid residues in length, and (ii) the amino acid entities of (a)-(f) are selected from Table 2. The total wt. % of (a)-(f) may be greater than the total wt. % of other amino acid entities in the composition.

In certain embodiments, the composition comprises: a) an ornithine (Orn)-amino acid entity, and b) an arginine (R)-amino acid entity, provided that: i) at least one amino acid entity is not provided as a peptide of more than 20 amino acid residues in length, and (ii) the amino acid entities of (a)-(b) are selected from Table 2. The total wt. % of (a)-(b) may be greater than the total wt. % of other amino acid entities in the composition.

In certain embodiments, the composition comprises: a) an glutamine (Q)-amino acid entity, and b) a glutamate (E)-amino acid entity, provided that: i) at least one amino acid entity is not provided as a peptide of more than 20 amino acid residues in length, and (ii) the amino acid entities of (a)-(b) are selected from Table 2. The total wt. % of (a)-(b) may be greater than the total wt. % of other amino acid entities in the composition.

In certain embodiments, the composition comprises: a) an methionine (M)-amino acid entity, and b) an cysteine (C)-amino acid entity, provided that: i) at least one amino acid entity is not provided as a peptide of more than 20 amino acid residues in length, and (ii) the amino acid entities of (a)-(b) are selected from Table 2. The total wt. % of (a)-(b) may be greater than the total wt. % of other amino acid entities in the composition.

In certain embodiments, the composition comprises: a) an methionine (M)-amino acid entity, and b) an N-acetylcysteine (NAC)-amino acid entity, provided that: i) at least one amino acid entity is not provided as a peptide of more than 20 amino acid residues in length, and (ii) the amino acid entities of (a)-(b) are selected from Table 2. The total wt. % of (a)-(b) may be greater than the total wt. % of other amino acid entities in the composition.

In certain embodiments, the composition comprises: a) a leucine (L)-amino acid entity, b) an isoleucine (I)-amino acid entity, c) a valine (V)-amino acid entity, and d) an aspartate (D)-amino acid entity, provided that: i) at least one amino acid entity is not provided as a peptide of more than 20 amino acid residues in length, and (ii) the amino acid entities of (a)-(d) are selected from Table 2. The total wt. % of (a)-(d) may be greater than the total wt. % of other amino acid entities in the composition.

In certain embodiments, the composition comprises: a) a leucine (L)-amino acid entity, b) an isoleucine (I)-amino acid entity, c) a valine (V)-amino acid entity, and d) an ornithine (Orn)-amino acid entity, provided that: i) at least one amino acid entity is not provided as a peptide of more than 20 amino acid residues in length, and (ii) the amino acid entities of (a)-(d) are selected from Table 2. The total wt. % of (a)-(d) may be greater than the total wt. % of other amino acid entities in the composition.

In some embodiments, at least 50 wt. % of the total wt., on a dry weight basis, e.g., when the composition is in powder form, of the composition is one or more amino acid entities in free form.

In some embodiments, the L-amino acid entity is selected from the group consisting of a precursor, a metabolite, and a derivative. In certain embodiments, the L-amino acid entity is selected from the group consisting of L-leucine, β-hydroxy-β-methylbutyrate (HMB), oxo-leucine, isovaleryl-CoA, D-leucine, and N-acetylleucine. In one embodiment, the L-amino acid entity is L-leucine. In another embodiment, the L-amino acid entity is HMB.

In some embodiments, the R-amino acid entity is selected from the group consisting of a precursor, a metabolite, and a derivative. In certain embodiments, the R-amino acid entity is selected from the group consisting of L-arginine, D-arginine, ornithine, argininosuccinate, citrulline, aspartate, glutamate, agmatine, and N-acetyl-arginine. In one embodiment, the R-amino acid entity is L-arginine. In one embodiment, the R-amino acid entity is creatine. In another embodiment, the R-amino acid entity is ornithine.

In some embodiments, the Q-amino acid entity is selected from the group consisting of a precursor, a metabolite, and a derivative. In certain embodiments, the Q-amino acid entity is selected from the group consisting of L-glutamine, glutamate, carbamoyl-P, glutamate, D-glutamine, and N-acetylglutamine. In one embodiment, the Q-amino acid entity is L-glutamine.

In some embodiments, the I-amino acid entity is selected from the group consisting of a salt, a precursor, a metabolite, and a derivative. In certain embodiments, the I-amino acid entity is selected from the group consisting of L-isoleucine, 2-oxo-3-methyl-valerate, threonine, 2-oxo-3-methyl-valerate, methylbutyl-CoA, D-isoleucine, and N-acetyl-isoleucine. In one embodiment, the I-amino acid entity is L-isoleucine.

In some embodiments, the V-amino acid entity is selected from the group consisting of a precursor, a metabolite, and a derivative. In certain embodiments, the V-amino acid entity is selected from the group consisting of L-valine, 2-oxo-valerate, isobutyl-CoA, 3-HIB-CoA, D-valine, and N-acetyl-valine. In one embodiment, the V-amino acid entity is L-valine.

In some embodiments, the H-amino acid entity is selected from the group consisting of a precursor, a metabolite, and a derivative. In certain embodiments, the H-amino acid entity is selected from the group consisting of L-histidine, histidinol, histidinal, ribose-5-phosphate, carnosine, histamine, urocanate, D-histidine, and N-acetyl-histidine. In certain embodiments, the H-amino acid entity is an amino acid, e.g., L-histidine. In certain embodiments, the H-amino acid entity is a precursor, e.g., histidinol, histidinal, or ribose-5-phosphate. In certain embodiments, the H-amino acid entity is a metabolite, e.g., carnosine, histamine, or urocanate. In certain embodiments, the H-amino acid entity is a derivative, e.g., D-histidine or N-acetyl-histidine.

In some embodiments, the derivative of an amino acid entity comprises an amino acid ester (e.g., an alkyl ester, e.g., an ethyl ester or a methyl ester of an amino acid entity) or a keto-acid.

In some embodiments, at least one amino acid entity is a free amino acid, e.g., one, two, three, four, five, six, seven, eight, nine, or more (e.g., all) amino acid entities are a free amino acid. In some embodiments, one or more of a leucine (L)-amino acid entity, an isoleucine (I)-amino acid entity, a valine (V)-amino acid entity, a glutamine (Q)-amino acid entity, a glutamic acid (E)-amino acid entity, an aspartic acid (D)-amino acid entity, a glycine (G)-amino acid entity, a serine (S)-amino acid entity, a methionine (M)-amino acid entity, a cysteine (C)-amino acid entity, an ornithine (Orn)-amino acid entity, an arginine (R)-amino acid entity, a histidine (H)-amino acid entity, a tyrosine (Y)-amino acid entity, and a tryptophan (W)-amino acid entity is a free amino acid.

In some embodiments, at least one amino acid entity is in a salt form, e.g., one, two, three, four, five, six, seven, eight, nine, or more (e.g., all) of the amino acid entities is in a salt form. In some embodiments, one or more of a leucine (L)-amino acid entity, an isoleucine (I)-amino acid entity, a valine (V)-amino acid entity, a glutamine (Q)-amino acid entity, a glutamic acid (E)-amino acid entity, an aspartic acid (D)-amino acid entity, a glycine (G)-amino acid entity, a serine (S)-amino acid entity, a methionine (M)-amino acid entity, a cysteine (C)-amino acid entity, an ornithine (Orn)-amino acid entity, an arginine (R)-amino acid entity, a histidine (H)-amino acid entity, a tyrosine (Y)-amino acid entity, and a tryptophan (W)-amino acid entity is in a salt form.

In some embodiments, the composition comprises a combination of 2 to 20 different amino acid entities, e.g., 5 to 15 different amino acid entities.

An aspect of the present disclosure provides a composition comprising free amino acids and one or more pharmaceutically acceptable excipients, such that the amino acids include leucine, isoleucine, valine, cysteine or N-acetylcysteine, and histidine; or such that the amino acids include glutamine, glutamate, aspartate, glycine and serine.

The disclosure also provides a composition comprising at least five different amino acid entities, in which the composition is capable of one, two, three, or all of:

a) activating mTORC1;

b) activating protein synthesis and/or inhibiting protein catabolism;

c) improving, e.g., increasing, insulin sensitivity or glucose tolerance; or

d) reducing inflammation;

provided that at least one amino acid entity is not a polypeptide of more than 20 amino acid residues in length.

The disclosure also provides a composition comprising at least five different amino acid entities, wherein said composition when administered to a subject results in one, two, three, or all of:

a) activating mTORC1;

b) activating protein synthesis and/or inhibiting protein catabolism;

c) improving insulin sensitivity or glucose tolerance; or

d) reducing inflammation;

provided that at least one amino acid entity is not a polypeptide of more than 20 amino acid residues in length.

In some embodiments, the protein synthesis is muscle protein synthesis. In some embodiments, the protein catabolism is muscle protein catabolism.

In some embodiments, the composition is capable of activating mTORC1 by at least 20%, 25%, 30%, 35%, 40%, 45%, 50%, 55%, 60%, 65%, 70%, 75%, 80%, 85%, 90%, 95%, or 99%, as detected using as an assay to measure mTORC1 substrate phosphorylation, e.g., P-rpS6 phosphorylation, e.g., an ELISA and/or cellular kinase assay, e.g., as described in Example 1, e.g., relative to a reference composition (e.g., an amino acid composition comprising L-leucine, L-isoleucine, L-valine; an amino acid composition comprising all 20 canonical amino acids).

In some embodiments, the composition is capable of phosphorylating an mTORC1 substrate e.g., P-rpS6 phosphorylation by at least 20%, 25%, 30%, 35%, 40%, 45%, 50%, 55%, 60%, 65%, 70%, 75%, 80%, 85%, 90%, 95%, or 99%, as detected using as assay to measure mTORC1 substrate phosphorylation, e.g., P-rpS6 phosphorylation, e.g., an ELISA and/or cellular kinase assay, e.g., as described in Example 1, e.g., relative to a reference composition (e.g., an amino acid composition comprising L-leucine, L-isoleucine, L-valine; an amino acid composition comprising all 20 canonical amino acids).

In some embodiments, the composition is capable of increasing myogenesis by at least 20%, 25%, 30%, 35%, 40%, 45%, 50%, 55%, 60%, 65%, 70%, 75%, 80%, 85%, 90%, 95%, or 99%, as detecting by counting myoblasts cells, e.g., human myotube cells or C2C12 cells, e.g., by a nuclear stain, e.g., a Hoechst stain, e.g., e.g., relative to a reference composition (e.g., an amino acid composition comprising L-leucine, L-isoleucine, L-valine; an amino acid composition comprising all 20 canonical amino acids).

In some embodiments, the composition is capable of increasing myoblast cell count by at least 20%, 25%, 30%, 35%, 40%, 45%, 50%, 55%, 60%, 65%, 70%, 75%, 80%, 85%, 90%, 95%, or 99%, as detecting by counting myoblasts cells, e.g., human myotube cells or C2C12 cells, e.g., by a nuclear stain, e.g., a Hoechst stain, e.g., relative to a reference composition (e.g., an amino acid composition comprising L-leucine, L-isoleucine, L-valine; an amino acid composition comprising all 20 canonical amino acids).

In some embodiments, the composition is capable of increasing myotube growth by at least 20%, 25%, 30%, 35%, 40%, 45%, 50%, 55%, 60%, 65%, 70%, 75%, 80%, 85%, 90%, 95%, or 99%, by detecting an increase of MyoD and/or Myogenin in, e.g., human myotube cells or C2C12 cells, e.g., as detected using as immunohistochemistry, e.g., relative to a reference composition (e.g., an amino acid composition comprising L-leucine, L-isoleucine, L-valine; an amino acid composition comprising all 20 canonical amino acids).

In some embodiments, the composition is capable of increasing MyoD and/or Myogenin by at least 20%, 25%, 30%, 35%, 40%, 45%, 50%, 55%, 60%, 65%, 70%, 75%, 80%, 85%, 90%, 95%, or 99%, as detecting by detecting an increase of MyoD and/or Myogenin in, e.g., human myotube cells or C2C12 cells, e.g., as detected using as immunohistochemistry, e.g., relative to a reference composition (e.g., an amino acid composition comprising L-leucine, L-isoleucine, L-valine; an amino acid composition comprising all 20 canonical amino acids).

In some embodiments, the composition is capable of activating protein synthesis and/or inhibiting protein catabolism by at least 25%, 30%, 35%, 40%, 45%, 50%, 55%, 60%, 65%, 70%, 75%, 80%, 85%, 90%, 95%, or 99%, as detected using an assay to measure Fractional Synthetic Rates (FSR) either in cultured myotubes or rodents, e.g., relative to a reference composition (e.g., an amino acid composition comprising L-leucine, L-isoleucine, L-valine; an amino acid composition comprising all 20 canonical amino acids).

In some embodiments, the composition is capable of inhibiting protein catabolism by at least 25%, 30%, 35%, 40%, 45%, 50%, 55%, 60%, 65%, 70%, 75%, 80%, 85%, 90%, 95%, or 99%, as detected using an assay to measure proteasomal activity, e.g., proteasomal activity in muscle tissue, e.g., proteasomal activity in skeletal muscle tissue, e.g., relative to a reference composition (e.g., an amino acid composition comprising L-leucine, L-isoleucine, L-valine; an amino acid composition comprising all 20 canonical amino acids).

In some embodiments, the composition is capable of improving insulin sensitivity or glucose tolerance by at least 25%, 30%, 35%, 40%, 45%, 50%, 55%, 60%, 65%, 70%, 75%, 80%, 85%, 90%, 95%, or 99%, as detected using as assay to measure insulin-stimulated glucose disposal or glucose-induced insulin secretion either in cultured myotubes or rodents, e.g., relative to a reference composition (e.g., an amino acid composition comprising L-leucine, L-isoleucine, L-valine; an amino acid composition comprising all 20 canonical amino acids).

In some embodiments, the composition is capable of reducing inflammation by at least 25%, 30%, 35%, 40%, 45%, 50%, 55%, 60%, 65%, 70%, 75%, 80%, 85%, 90%, 95%, or 99%, as detected using as assay to measure cytokine or collagen production either in cells or in vivo, e.g., relative to a reference composition (e.g., an amino acid composition comprising L-leucine, L-isoleucine, L-valine; an amino acid composition comprising all 20 canonical amino acids).

In some embodiments, the composition is capable of increasing protein synthesis by at least 25%, 30%, 35%, 40%, 45%, 50%, 55%, 60%, 65%, 70%, 75%, 80%, 85%, 90%, 95%, or 99%, as detected using, e.g., the method of Example 2, e.g., relative to a reference composition (e.g., an amino acid composition comprising L-leucine, L-isoleucine, L-valine; an amino acid composition comprising all 20 canonical amino acids).

In some embodiments, the composition is capable of improving myogenesis and/or myotube differentiation, e.g., improving (e.g., increasing) myotube fusion index, by at least 25%, 30%, 35%, 40%, 45%, 50%, 55%, 60%, 65%, 70%, 75%, 80%, 85%, 90%, 95%, or 99%, as detected using, e.g., the method of Example 1, e.g., relative to a reference composition (e.g., an amino acid composition comprising L-leucine, L-isoleucine, L-valine; an amino acid composition comprising all 20 canonical amino acids).

In some embodiments, the reference composition comprises a single amino acid entity, e.g., leucine (L)-amino acid entity, an isoleucine (I)-amino acid entity, a valine (V)-amino acid entity, a glutamine (Q)-amino acid entity, a glutamic acid (E)-amino acid entity, an aspartic acid (D)-amino acid entity, a glycine (G)-amino acid entity, a serine (S)-amino acid entity, a methionine (M)-amino acid entity, a cysteine (C)-amino acid entity, an ornithine (Orn)-amino acid entity, an arginine (R)-amino acid entity, a histidine (H)-amino acid entity, a tyrosine (Y)-amino acid entity, or a tryptophan (W)-amino acid entity, each assayed separately as a free amino acid, or a combination of amino acid entities. In certain embodiments, the reference composition comprises a vehicle (e.g., PBS or saline).

### Production of the Amino Acid Compositions

Amino acids used to make the compositions may be agglomerated, and/or instantized to aid in dispersal and/or solubilization.

The amino acid compositions of the present disclosure may be made using amino acids and amino acid derivatives from the following sources, or other sources may used: e.g., FUSI-BCAA™ Instantized Blend (L-Leucine, L-Isoleucine and L-Valine in 2:1:1 weight ratio), FUSIL™ Instantized L-Leucine, L-Arginine HCl, L-Glutamine and other amino acids may be obtained from Ajinomoto Co., Inc; N-acetyl-cysteine may be obtained from Spectrum Chemical.

To produce the amino acid compositions of the instant disclosure, the following general steps may be used: the starting materials (individual amino acids and excipients) may be blended in a blending unit, followed by verification of blend uniformity and amino acid content, and filling of the blended powder into stick packs or other unit dosage form. The content of stick packs or other unit dosage forms may be dispersed in water at time of use for oral administration. Examples of the methods of production of amino acid compositions are disclosed in U.S. patent application Ser. No. 16/446,171, filed Jun. 19, 2019, entitled “METHODS OF MANUFACTURING AMINO ACID COMPOSITIONS,” which is incorporated herein by reference in its entirety.

### Formulations

The pharmaceutical compositions of the present disclosure may be in a form suitable for oral use (for example as tablets, lozenges, hard or soft capsules, aqueous or oily suspensions, emulsions, dispersible powders or granules, syrups or elixirs, medical food products, nutraceuticals), for topical use (for example as creams, ointments, gels, or aqueous or oily solutions or suspensions), for administration by inhalation (for example as finely divided powder) for parenteral administration (for example as a sterile aqueous or oily solution for intravenous, subcutaneous, intramuscular dosing or as a suppository for rectal dosing) or for enteral administration (for example via tube feeding).

### Excipients

The amino acid compositions of the present disclosure may be compounded or formulated with one or more excipients. Non-limiting examples of suitable excipients include a tastant, a flavorant, a buffering agent, a preservative, a stabilizer, a binder, a compaction agent, a lubricant, a dispersion enhancer, a disintegration agent, a flavoring agent, a sweetener, and a coloring agent. U.S. patent application Ser. No. 16/446,192, filed Jun. 19, 2019, entitled “COMPOSITIONS FOR THERAPY AND HEALTH CONTAINING AMINO ACIDS WITH BITTER TASTE,” which is incorporated herein by reference in its entirety describes suitable excipients for inclusion in compositions of the invention.

In some embodiments, the excipient comprises a buffering agent. Non-limiting examples of suitable buffering agents include citric acid, sodium citrate, magnesium carbonate, magnesium bicarbonate, calcium carbonate, and calcium bicarbonate.

In some embodiments, the excipient comprises a preservative. Non-limiting examples of suitable preservatives include antioxidants, such as alpha-tocopherol and ascorbate, and antimicrobials, such as parabens, chlorobutanol, and phenol.

In some embodiments, the composition comprises a binder as an excipient. Non-limiting examples of suitable binders include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides, and combinations thereof.

In some embodiments, the composition comprises a lubricant as an excipient. Non-limiting examples of suitable lubricants include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil.

In some embodiments, the composition comprises a dispersion enhancer as an excipient. Non-limiting examples of suitable dispersants include starch, alginic acid, polyvinylpyrrolidones, guar gum, kaolin, xanthan gum, bentonite, purified wood cellulose, sodium starch glycolate, isoamorphous silicate, and microcrystalline cellulose as high HLB emulsifier surfactants.

In some embodiments, the composition comprises a disintegrant as an excipient. In some embodiments, the disintegrant is a non-effervescent disintegrant. Non-limiting examples of suitable non-effervescent disintegrants include starches such as corn starch, potato starch, pregelatinized and modified starches thereof, sweeteners, clays, such as bentonite, microcrystalline cellulose, alginates, sodium starch glycolate, gums such as agar, guar, locust bean, karaya, pectin, and tragacanth. In some embodiments, the disintegrant is an effervescent disintegrant. Non-limiting examples of suitable effervescent disintegrants include sodium bicarbonate in combination with citric acid, and sodium bicarbonate in combination with tartaric acid.

In some embodiments, the excipient comprises a flavoring agent. Flavoring agents can be chosen from synthetic flavor oils and flavoring aromatics; natural oils; extracts from plants, leaves, flowers, and fruits; and combinations thereof. In some embodiments, the flavoring agent is selected from cinnamon oils; oil of wintergreen; peppermint oils; clover oil; hay oil; anise oil; eucalyptus; vanilla; citrus oil such as lemon oil, orange oil, grape and grapefruit oil; and fruit essences including apple, peach, pear, strawberry, raspberry, cherry, plum, pineapple, and apricot.

In some embodiments, the excipient comprises a sweetener. Non-limiting examples of suitable sweeteners include glucose (corn syrup), dextrose, invert sugar, fructose, and mixtures thereof (when not used as a carrier); saccharin and its various salts such as the sodium salt; dipeptide sweeteners such as aspartame; dihydrochalcone compounds, glycyrrhizin; Stevia Rebaudiana (Stevioside); chloro derivatives of sucrose such as sucralose; and sugar alcohols such as sorbitol, mannitol, xylitol, and the like. Also contemplated are hydrogenated starch hydrolysates and the synthetic sweetener 3,6-dihydro-6-methyl-1,2,3-oxathiazin-4-one-2,2-dioxide, particularly the potassium salt (acesulfame-K), and sodium and calcium salts thereof.

In some embodiments, the composition comprises a coloring agent. Non-limiting examples of suitable color agents include food, drug and cosmetic colors (FD&C), drug and cosmetic colors (D&C), and external drug and cosmetic colors (Ext. D&C). The coloring agents can be used as dyes or their corresponding lakes.

In some embodiments, the composition comprises a wetting agent. Non-limiting examples of suitable wetting agents include one or more lecithin such as Alcolec Lecithin F100 or a substantially equivalent lecithin, Alcolec Lecithin 40P or a substantially equivalent lecithin, Lipoid 20S Lecithin or a substantially equivalent lecithin; and one or more poloxamer such as poloxamer P331 or a substantially equivalent poloxamer.

In some embodiments, the composition comprises an adsorbent. Non-limiting examples of suitable adsorbents include SiO₂, Mg silicate, Ca silicate, Talc, Ca carbonate, Magnesium carbonate, MgO, Ca sulfate, CaCl₂, Al metal silicate, anhydrous Si acid, Mg Aluminum Silicate, Microcrystalline cellulose, Sodium carboxymethylcellulose, and Calcium carboxymethylcellulose. In some embodiments, the composition comprises an adsorbent selected from SiO₂, mesoporous and/or colloidal SiO₂, and Aerosil 300 SiO₂ or a substantially equivalent SiO₂.

Particular excipients may include one or more of: citric acid, lecithin, (e.g., Alcolec F100, Alcolec 40P, or Lipoid 20S), sweeteners (e.g., sucralose, sucralose micronized NF, acesulfame potassium (e.g., Ace-K)), a dispersion enhancer (e.g., xanthan gum (e.g., Ticaxan Rapid-3)), flavorings (e.g., vanilla custard #4306, Nat Orange WONF #1326, lime 865.0032U, and lemon 862.2169U), a bitterness masking agent (e.g., 936.2160U), and natural or artificial colorings (e.g., FD&C Yellow 6).

### Methods of Treatment

The composition as described herein can be administered to improve, e.g., enhance, muscle function, muscle mass, muscle protein synthesis and/or myogenesis, e.g., in a patient with a muscle disease or disorder. The present disclosure also provides a method for treating one, two, three, four, five, six, seven, eight, nine, or more (e.g., all) physiological symptoms selected from immobilization, malnutrition, fasting, aging, autophagy, reduced protein synthesis, anabolic resistance, neuromuscular junction integrity, insulin resistance, decreased mitochondrial biogenesis, anaplerosis, or an energy deficit. The method includes administering to a subject in need thereof an effective amount of the composition. In some embodiments, the subject has a rare muscle disease. In some embodiments, the subject has muscle atrophy, sarcopenia, muscle deterioration, muscle decay, cachexia, drug-induced myopathy, muscular dystrophy, or myopenia. In some embodiments, the composition as described herein can be administered or used as a dietary supplement for a healthy subject or to potentiate the benefits of exercise in a subject.

In some embodiments, the subject has a muscle disease or disorder. In some embodiments, the muscle disease or disorder is a dystrophy. In some embodiments, the muscle disease or disorder is a myotonic dystrophy. In some embodiments, the muscle disease or disorder is DM1.

In some embodiments, the muscle disease or disorder is a drug-induced myopathy. In some embodiments, the muscle disease or disorder is a statin-induced myopathy. In some embodiments, the muscle disease or disorder is a steroid-induced myopathy. In some embodiments, the muscle disease or disorder is an immunosuppressant-induced myopathy. In some embodiments, the muscle disease or disorder is a chemotherapeutic-induced myopathy. In some embodiments, the muscle disease or disorder is an alcohol-induced myopathy.

In some embodiments, the subject has a fracture or other trauma. In some embodiments, the subject has a drug-induced myopathy. In some embodiments, the subject has a statin-induced myopathy. In some embodiments, the subject has a steroid-induced myopathy. In some embodiments, the subject has an immunosuppressant-induced myopathy. In some embodiments, the subject has a chemotherapeutic-induced myopathy. In some embodiments, the subject has an alcohol-induced myopathy. In some embodiments, the method includes administering to a subject in need thereof an effective amount of the composition to treat immobilization. In some embodiments, the subject has a rare muscle disease. In some embodiments, the subject has muscle atrophy, sarcopenia, muscle deterioration, muscle decay, cachexia, drug-induced myopathy, muscular dystrophy, or myopenia.

In some embodiments, the method includes administering to a subject in need thereof an effective amount of the composition to treat malnutrition. In some embodiments, the subject has a rare muscle disease. In some embodiments, the subject has muscle atrophy, sarcopenia, muscle deterioration, muscle decay, cachexia, drug-induced myopathy, muscular dystrophy, or myopenia.

In some embodiments, the method includes administering to a subject in need thereof an effective amount of the composition to treat fasting. In some embodiments, the subject has a rare muscle disease. In some embodiments, the subject has muscle atrophy, sarcopenia, muscle deterioration, muscle decay, cachexia, drug-induced myopathy, muscular dystrophy, or myopenia.

In some embodiments, the method includes administering to a subject in need thereof an effective amount of the composition to treat aging. In some embodiments, the subject has a rare muscle disease. In some embodiments, the subject has muscle atrophy, sarcopenia, muscle deterioration, muscle decay, cachexia, drug-induced myopathy, muscular dystrophy, or myopenia.

In some embodiments, the method includes administering to a subject in need thereof an effective amount of the composition to treat autophagy. In some embodiments, the subject has a rare muscle disease. In some embodiments, the subject has muscle atrophy, sarcopenia, muscle deterioration, muscle decay, cachexia, drug-induced myopathy, muscular dystrophy, or myopenia.

In some embodiments, the method includes administering to a subject in need thereof an effective amount of the composition to treat reduced protein synthesis. In some embodiments, the subject has a rare muscle disease. In some embodiments, the subject has muscle atrophy, sarcopenia, muscle deterioration, muscle decay, cachexia, drug-induced myopathy, muscular dystrophy, or myopenia.

In some embodiments, the method includes administering to a subject in need thereof an effective amount of the composition to treat anabolic resistance. In some embodiments, the subject has a rare muscle disease. In some embodiments, the subject has muscle atrophy, sarcopenia, muscle deterioration, muscle decay, cachexia, drug-induced myopathy, muscular dystrophy, or myopenia.

In some embodiments, the method includes administering to a subject in need thereof an effective amount of the composition to treat junction integrity (e.g., neuromuscular junction integrity). In some embodiments, the subject has a rare muscle disease. In some embodiments, the subject has muscle atrophy, sarcopenia, muscle deterioration, muscle decay, cachexia, drug-induced myopathy, muscular dystrophy, or myopenia.

In some embodiments, the method includes administering to a subject in need thereof an effective amount of the composition to treat insulin resistance. In some embodiments, the subject has a rare muscle disease. In some embodiments, the subject has muscle atrophy, sarcopenia, muscle deterioration, muscle decay, cachexia, drug-induced myopathy, muscular dystrophy, or myopenia.

In some embodiments, the method includes administering to a subject in need thereof an effective amount of the composition to treat decreased mitochondrial biogenesis. In some embodiments, the subject has a rare muscle disease. In some embodiments, the subject has muscle atrophy, sarcopenia, muscle deterioration, muscle decay, cachexia, drug-induced myopathy, muscular dystrophy, or myopenia.

In some embodiments, the method includes administering to a subject in need thereof an effective amount of the composition to treat anaplerosis. In some embodiments, the subject has a rare muscle disease. In some embodiments, the subject has muscle atrophy, sarcopenia, muscle deterioration, muscle decay, cachexia, drug-induced myopathy, muscular dystrophy, or myopenia.

In some embodiments, the method includes administering to a subject in need thereof an effective amount of the composition to treat an energy deficit. In some embodiments, the subject has a rare muscle disease. In some embodiments, the subject has muscle atrophy, sarcopenia, muscle deterioration, muscle decay, cachexia, drug-induced myopathy, muscular dystrophy, or myopenia.

The present disclosure also provides methods for enhancing muscle function that include administering to a subject in need thereof an effective amount of a composition including defined amino acid components. In some embodiments, the subject has or is identified as having decreased muscle function due to aging, injury, atrophy, infection, or disease. In some embodiments, the composition reduces muscle atrophy in the subject.

In some embodiments, the subject has or is identified as having muscle deterioration, decay, atrophy, cachexia, sarcopenia, drug-induced myopathy, muscular dystrophy, or myopenia. In some embodiments, the subject is a human. In some embodiments, the subject has not received prior treatment with a composition including defined amino acid components (e.g., a naïve subject).

In some embodiments, the subject has or is identified as having muscle deterioration. In some embodiments, the subject is a human. In some embodiments, the subject has not received prior treatment with a composition including defined amino acid components (e.g., a naïve subject).

In some embodiments, the subject has or is identified as having muscle decay. In some embodiments, the subject is a human. In some embodiments, the subject has not received prior treatment with a composition including defined amino acid components (e.g., a naïve subject).

In some embodiments, the subject has or is identified as having muscle atrophy. In some embodiments, the subject is a human. In some embodiments, the subject has not received prior treatment with a composition including defined amino acid components (e.g., a naïve subject).

In some embodiments, the subject has or is identified as having cachexia. In some embodiments, the subject is a human. In some embodiments, the subject has not received prior treatment with a composition including defined amino acid components (e.g., a naïve subject).

In some embodiments, the subject has or is identified as having sarcopenia. In some embodiments, the subject is a human. In some embodiments, the subject has not received prior treatment with a composition including defined amino acid components (e.g., a naïve subject).

In some embodiments, the subject has or is identified as having drug-induced myopathy. In some embodiments, the subject is a human. In some embodiments, the subject has not received prior treatment with a composition including defined amino acid components (e.g., a naïve subject).

In some embodiments, the subject has or is identified as having muscular dystrophy. In some embodiments, the subject is a human. In some embodiments, the subject has not received prior treatment with a composition including defined amino acid components (e.g., a naïve subject).

In some embodiments, the subject has or is identified as having myopenia. In some embodiments, the subject is a human. In some embodiments, the subject has not received prior treatment with a composition including defined amino acid components (e.g., a naïve subject).

In some embodiments, the subject has muscle weakness, e.g., muscle weakness of one, two, three, or more (e.g., all) of skeletal muscle, cardiac muscle, or smooth muscle. In certain embodiments, the subject has muscle weakness in one, two, three, four, five, six, or more (e.g., all) of a neck muscle, a torso muscle, an arm muscle, a shoulder muscle, a hand muscle, a leg muscle, or a foot muscle.

In some embodiments, the subject has had a surgery, e.g., rotator cuff surgery, knee surgery, or hip surgery, or has worn a cast prior to administration of the composition. In an embodiment, the subject has had rotator cuff surgery prior to administration of the composition. In an embodiment, the subject has had a knee surgery prior to administration of the composition. In an embodiment, the subject has had a hip surgery prior to administration of the composition. In an embodiment, the subject has worn a cast prior to administration of the composition.

In some embodiments, the subject has perceived muscle weakness, e.g., chronic fatigue syndrome.

In some embodiments, the subject has a cancer-associated muscle weakness.

In some embodiments, the subject has a neuromuscular disorder, e.g., myasthenia gravis or Lambert-Eaton myasthenic syndrome.

In some embodiments, the subject has muscular dystrophy, e.g., Duchenne muscular dystrophy, Becker muscular dystrophy, facioscapulohumeral muscular dystrophy, or myotonic dystrophy. In some embodiments, the subject has inflammatory myopathy, e.g., polymyositis or dermatomyositis.

In some embodiments, the subject has one, two, or more (e.g., all) of low sodium levels (e.g., hyponatremia), low potassium levels (e.g., hypokalemia), or a calcium deficiency or relatively high calcium levels (e.g., hypercalcemia).

In some embodiments, the subject has muscle weakness associated with nerve damage, e.g., neuralgia or peripheral neuropathy. In some embodiments, the subject has a bone weakness disease, e.g., osteomalacia, osteogenesis imperfecta, rickets, or hypophosphatasia.

In some embodiments, the subject has experienced a stroke or a transient ischemic attack. In some embodiments, the subject has an autoimmune disease, e.g., Graves' disease.

In some embodiments, the subject has hypothyroidism. In some embodiments, the subject has amyotrophic lateral sclerosis (ALS).

In some embodiments, administering the composition results in an improvement in one or more metabolic symptoms in the subject. In certain embodiments, the one or more metabolic symptoms is selected from the following: mTORC1 activation; improved insulin sensitivity; activation of muscle protein synthesis; scavenging of reactive oxygen species (ROS); decreased inflammation; inhibition catabolism; ammonia detoxification; and decreased fibrosis progression.

In some embodiments, the composition reduces muscle atrophy.

In some embodiments, the composition results in anabolism and catabolism of muscle tissue in the subject.

In some embodiments, administering the composition results in mTORC1 activation in the subject. In some embodiments, the composition also reduces muscle atrophy.

In some embodiments, administering the composition results in improved insulin sensitivity in the subject. In some embodiments, the composition also reduces muscle atrophy.

In some embodiments, administering the composition results in activation of muscle protein synthesis in the subject. In some embodiments, the composition also reduces muscle atrophy.

In some embodiments, administering the composition results in scavenging of reactive oxygen species (ROS) in the subject. In some embodiments, the composition also reduces muscle atrophy.

In some embodiments, administering the composition results in decreased inflammation in the subject. In some embodiments, the composition also reduces muscle atrophy.

In some embodiments, administering the composition results inhibited catabolism in the subject. In some embodiments, the composition also reduces muscle atrophy.

In some embodiments, administering the composition results in ammonia detoxification in the subject. In some embodiments, the composition also reduces muscle atrophy.

In some embodiments, administering the composition results in decreased fibrosis progression in the subject. In some embodiments, the composition also reduces muscle atrophy.

In some embodiments, the composition results in an improvement in one or both of muscle loss or muscle function related to one or both of immobilization or muscle disuse following injury in a subject. In some embodiments, the subject has had a surgery, e.g., rotator cuff surgery, knee surgery, or hip surgery, or has worn a cast, prior to administration of the composition. In some embodiments, the subject has had a hip fracture-related myopenia. In some embodiments, the subject has had a joint replacement. In some embodiments, the subject has had an injury repair surgery.

In some embodiments, the subject has ventilator-induced diaphragmatic dystrophy or ventilator-induced diaphragmatic dysfunction. In some embodiments, the subject has had one or both of ICU-acquired or burns-related myopathies.

In some embodiments, the subject has disease-related cachexia, e.g., a disease-related cachexia selected from chronic obstructive pulmonary disease (COPD), congestive heart failure (CHF), chronic kidney disease (CKD), and cancer.

In some embodiments, the composition is administered with a second agent.

The present disclosure also provides a method for reducing muscle atrophy comprising administering to a subject in need thereof an effective amount of a composition described herein.

The present disclosure also provides a composition described herein for use as a medicament.

The present disclosure provides a composition described herein for use as a medicament in enhancing muscle function.

The present disclosure provides a composition described herein for use as a medicament for treating one or more symptoms selected from the group consisting of immobilization, malnutrition, fasting, aging, autophagy, reduced protein synthesis, anabolic resistance, neuromuscular junction integrity, insulin resistance, decreased mitochondrial biogenesis, and anaplerosis.

The present disclosure provides a composition described herein for use in the manufacture of a medicament for enhancing muscle function. The present disclosure provides a use of a composition for the manufacture of a medicament for treating one or more symptoms selected from the group consisting of immobilization, malnutrition, fasting, aging, autophagy, reduced protein synthesis, anabolic resistance, neuromuscular junction integrity, insulin resistance, decreased mitochondrial biogenesis, and anaplerosis.

In some embodiments, the disclosure is directed to a method of increasing protein synthesis (e.g., in a cell, tissue, or a subject), the method comprising administering a composition comprising: a leucine (L)-amino acid entity, an isoleucine (I)-amino acid entity, a valine (V)-amino acid entity, an aspartic acid (D)-amino acid entity, a methionine (M)-amino acid entity, a cysteine (C)-amino acid entity, an arginine (R)-amino acid entity, a histidine (H)-amino acid entity, a tyrosine (Y)-amino acid entity, and a tryptophan (W)-amino acid entity.

In some embodiments, the disclosure is directed to a method of increasing protein synthesis (e.g., in a cell, tissue, or a subject), the method comprising administering a composition comprising a leucine (L)-amino acid entity, an isoleucine (I)-amino acid entity, a valine (V)-amino acid entity, a glutamine (Q)-amino acid entity, a glutamic acid (E)-amino acid entity, an aspartic acid (D)-amino acid entity, a glycine (G)-amino acid entity, a serine (S)-amino acid entity, a methionine (M)-amino acid entity, a cysteine (C)-amino acid entity, an ornithine (Orn)-amino acid entity, an arginine (R)-amino acid entity, a histidine (H)-amino acid entity, a tyrosine (Y)-amino acid entity, and a tryptophan (W)-amino acid entity.

In some embodiments, the disclosure is directed to a method of increasing protein synthesis (e.g., in a cell, tissue, or a subject), the method comprising administering a composition comprising a leucine (L)-amino acid entity, an isoleucine (I)-amino acid entity, a valine (V)-amino acid entity, a glutamic acid (E)-amino acid entity, an aspartic acid (D)-amino acid entity, a serine (S)-amino acid entity, a cysteine (C)-amino acid entity, and a histidine (H)-amino acid entity.

In some embodiments, the disclosure is directed to a method of increasing protein synthesis (e.g., in a cell, tissue, or a subject), the method comprising administering a composition comprising a leucine (L)-amino acid entity, an isoleucine (I)-amino acid entity, a valine (V)-amino acid entity, methionine (M)-amino acid entity, a cysteine (C)-amino acid entity, an arginine (R)-amino acid entity, a histidine (H)-amino acid entity, a tyrosine (Y)-amino acid entity, and a tryptophan (W)-amino acid entity.

In some embodiments, the disclosure is directed to a method of increasing protein synthesis (e.g., in a cell, tissue, or a subject), the method comprising administering a composition comprising a leucine (L)-amino acid entity, an isoleucine (I)-amino acid entity, a valine (V)-amino acid entity, a glutamic acid (E)-amino acid entity, an aspartic acid (D)-amino acid entity, a serine (S)-amino acid entity, a cysteine (C)-amino acid entity, an ornithine (Orn)-amino acid entity, and a histidine (H)-amino acid entity.

In some embodiments, the disclosure is directed to a method of increasing protein synthesis (e.g., in a cell, tissue, or a subject), the method comprising administering a composition comprising a leucine (L)-amino acid entity, an isoleucine (I)-amino acid entity, a valine (V)-amino acid entity, an aspartic acid (D)-amino acid entity, a cysteine (C)-amino acid entity, and an ornithine (Orn)-amino acid entity.

In some embodiments, the disclosure is directed to a method of increasing protein synthesis (e.g., in a cell, tissue, or a subject), the method comprising administering a composition comprising a leucine (L)-amino acid entity, an isoleucine (I)-amino acid entity, a valine (V)-amino acid entity, a glutamic acid (E)-amino acid entity, an aspartic acid (D)-amino acid entity, a serine (S)-amino acid entity, a cysteine (C)-amino acid entity, an ornithine (Orn)-amino acid entity, a histidine (H)-amino acid entity, a tyrosine (Y)-amino acid entity, and a tryptophan (W)-amino acid entity.

In some embodiments, the disclosure is directed to a method of increasing protein synthesis (e.g., in a cell, tissue, or a subject), the method comprising administering a composition comprising a leucine (L)-amino acid entity, an isoleucine (I)-amino acid entity, a valine (V)-amino acid entity, a glutamine (Q)-amino acid entity, a glycine (G)-amino acid entity, a serine (S)-amino acid entity, a methionine (M)-amino acid entity, a cysteine (C)-amino acid entity, an arginine (R)-amino acid entity, a histidine (H)-amino acid entity, a tyrosine (Y)-amino acid entity, and a tryptophan (W)-amino acid entity.

In some embodiments, the disclosure is directed to a method of increasing protein synthesis (e.g., in a cell, tissue, or a subject), the method comprising administering a composition comprising a glutamine (Q)-amino acid entity, a glutamic acid (E)-amino acid entity, an aspartic acid (D)-amino acid entity, a glycine (G)-amino acid entity, and a serine (S)-amino acid entity.

In some embodiments, the disclosure is directed to a method of increasing protein synthesis (e.g., in a cell, tissue, or a subject), the method comprising administering a composition comprising a leucine (L)-amino acid entity, an isoleucine (I)-amino acid entity, a valine (V)-amino acid entity, an ornithine (Orn)-amino acid entity, and an arginine (R)-amino acid entity.

In some embodiments, the disclosure is directed to a method of increasing protein synthesis (e.g., in a cell, tissue, or a subject), the method comprising administering a composition comprising a leucine (L)-amino acid entity, an isoleucine (I)-amino acid entity, a valine (V)-amino acid entity, a glutamic acid (E)-amino acid entity, an aspartic acid (D)-amino acid entity, a serine (S)-amino acid entity, an ornithine (Orn)-amino acid entity, and a histidine (H)-amino acid entity.

In some embodiments, the disclosure is directed to a method of increasing protein synthesis (e.g., in a cell, tissue, or a subject), the method comprising administering a composition comprising a leucine (L)-amino acid entity, an isoleucine (I)-amino acid entity, a valine (V)-amino acid entity, a methionine (M)-amino acid entity, and a cysteine (C)-amino acid entity.

In some embodiments, the disclosure is directed to a method of increasing protein synthesis (e.g., in a cell, tissue, or a subject), the method comprising administering a composition comprising a leucine (L)-amino acid entity, an isoleucine (I)-amino acid entity, a valine (V)-amino acid entity, a glutamine (Q)-amino acid entity, a glutamic acid (E)-amino acid entity, an aspartic acid (D)-amino acid entity, a glycine (G)-amino acid entity, a serine (S)-amino acid entity, a cysteine (C)-amino acid entity, and an ornithine (Orn)-amino acid entity.

In some embodiments, the disclosure is directed to a method of increasing protein synthesis (e.g., in a cell, tissue, or a subject), the method comprising administering a composition comprising a glutamine (Q)-amino acid entity, a glutamic acid (E)-amino acid entity, an aspartic acid (D)-amino acid entity, a glycine (G)-amino acid entity, an ornithine (Orn)-amino acid entity, and an arginine (R)-amino acid entity.

In some embodiments, the disclosure is directed to a method of increasing protein synthesis (e.g., in a cell, tissue, or a subject), the method comprising administering a composition comprising: a leucine (L)-amino acid entity, an isoleucine (I)-amino acid entity, a valine (V)-amino acid entity, a cysteine (C)-amino acid entity or a NAC entity, and a histidine (H)-amino acid entity.

In some embodiments, the disclosure is directed to a method of increasing protein synthesis (e.g., in a cell, tissue, or a subject), the method comprising administering a composition comprising: a leucine (L)-amino acid entity, an isoleucine (I)-amino acid entity, a valine (V)-amino acid entity, a cysteine (C)-amino acid entity, and an ornithine (Orn)-amino acid entity.

In some embodiments, the disclosure is directed to a method of improving (e.g., increasing) myotube fusion index (fusion index) (e.g., in a cell, tissue, or a subject), the method comprising administering a composition comprising a methionine (M)-amino acid entity and a cysteine (C)-amino acid entity.

In some embodiments, the disclosure is directed to a method of improving (e.g., increasing) myotube fusion index (fusion index) or myogenesis (e.g., in a cell, tissue, or a subject), the method comprising administering a composition comprising a leucine (L)-amino acid entity, an isoleucine (I)-amino acid entity, a valine (V)-amino acid entity, a glutamine (Q)-amino acid entity, a glutamic acid (E)-amino acid entity, an aspartic acid (D)-amino acid entity, a glycine (G)-amino acid entity, a serine (S)-amino acid entity, a cysteine (C)-amino acid entity, and an ornithine (Orn)-amino acid entity.

In some embodiments, the disclosure is directed to a method of improving (e.g., increasing) myotube fusion index (fusion index) or myogenesis (e.g., in a cell, tissue, or a subject), the method comprising administering a composition comprising a leucine (L)-amino acid entity, an isoleucine (I)-amino acid entity, a valine (V)-amino acid entity, a glutamic acid (E)-amino acid entity, an aspartic acid (D)-amino acid entity, a serine (S)-amino acid entity, a cysteine (C)-amino acid entity, and a histidine (H)-amino acid entity.

In some embodiments, the disclosure is directed to a method of improving (e.g., increasing) myotube fusion index (fusion index) or myogenesis (e.g., in a cell, tissue, or a subject), the method comprising administering a composition comprising: a leucine (L)-amino acid entity, an isoleucine (I)-amino acid entity, a valine (V)-amino acid entity, a glutamic acid (E)-amino acid entity, an aspartic acid (D)-amino acid entity, and a glycine (G)-amino acid entity.

In some embodiments, the disclosure is directed to a method of improving (e.g., increasing) myotube fusion index (fusion index) or myogenesis (e.g., in a cell, tissue, or a subject), the method comprising administering a composition to comprising a leucine (L)-amino acid entity, an isoleucine (I)-amino acid entity, a valine (V)-amino acid entity, an ornithine (Orn)-amino acid entity, and an arginine (R)-amino acid entity.

In some embodiments, the disclosure is directed to a method of improving (e.g., increasing) myotube fusion index (fusion index) or myogenesis (e.g., in a cell, tissue, or a subject), the method comprising administering a composition comprising a leucine (L)-amino acid entity, an isoleucine (I)-amino acid entity, a valine (V)-amino acid entity, an aspartic acid (D)-amino acid entity, a cysteine (C)-amino acid entity, and an ornithine (Orn)-amino acid entity.

In some embodiments, the disclosure is directed to a method of improving (e.g., increasing) myotube fusion index (fusion index) or myogenesis (e.g., in a cell, tissue, or a subject), the method comprising administering a composition comprising a glutamine (Q)-amino acid entity and a glutamic acid (E)-amino acid entity.

In some embodiments, the disclosure is directed to a method of improving (e.g., increasing) myotube fusion index (fusion index) or myogenesis (e.g., in a cell, tissue, or a subject), the method comprising administering a composition comprising a leucine (L)-amino acid entity, an isoleucine (I)-amino acid entity, a valine (V)-amino acid entity, a cysteine (C)-amino acid entity, and an ornithine (Orn)-amino acid entity.

In some embodiments, the disclosure is directed to a method of improving (e.g., increasing) myotube fusion index (fusion index) or myogenesis (e.g., in a cell, tissue, or a subject), the method comprising administering a composition comprising an ornithine (Orn)-amino acid entity and an arginine (R)-amino acid entity.

In some embodiments, the disclosure is directed to a method of improving (e.g., increasing) myotube fusion index (fusion index) or myogenesis (e.g., in a cell, tissue, or a subject), the method comprising administering a composition comprising a leucine (L)-amino acid entity, an isoleucine (I)-amino acid entity, a valine (V)-amino acid entity, a glutamic acid (E)-amino acid entity, an aspartic acid (D)-amino acid entity, a serine (S)-amino acid entity, a cysteine (C)-amino acid entity, an ornithine (Orn)-amino acid entity, and a histidine (H)-amino acid entity.

In some embodiments, the disclosure is directed to a method of improving (e.g., increasing) myotube fusion index (fusion index) or myogenesis (e.g., in a cell, tissue, or a subject), the method comprising administering a composition comprising a glutamine (Q)-amino acid entity, a glutamic acid (E)-amino acid entity, an aspartic acid (D)-amino acid entity, a glycine (G)-amino acid entity, an ornithine (Orn)-amino acid entity and an arginine (R)-amino acid entity.

In some embodiments, the disclosure is directed to a method of improving (e.g., increasing) myotube fusion index (fusion index) or myogenesis (e.g., in a cell, tissue, or a subject), the method comprising administering a composition comprising a leucine (L)-amino acid entity, an isoleucine (I)-amino acid entity, a valine (V)-amino acid entity, and an aspartic acid (D)-amino acid entity.

In some embodiments, the disclosure is directed to a method of improving (e.g., increasing) myotube fusion index (fusion index) or myogenesis (e.g., in a cell, tissue, or a subject), the method comprising administering a composition comprising a leucine (L)-amino acid entity, an isoleucine (I)-amino acid entity, a valine (V)-amino acid entity, an aspartic acid (D)-amino acid entity, a methionine (M)-amino acid entity, a cysteine (C)-amino acid entity, an arginine (R)-amino acid entity, a histidine (H)-amino acid entity, a tyrosine (Y)-amino acid entity, and a tryptophan (W)-amino acid entity.

In some embodiments, the disclosure is directed to a method of improving (e.g., increasing) myotube fusion index (fusion index) or myogenesis (e.g., in a cell, tissue, or a subject), the method comprising administering a composition comprising a leucine (L)-amino acid entity, an isoleucine (I)-amino acid entity, a valine (V)-amino acid entity, a glutamine (Q)-amino acid entity, a glutamic acid (E)-amino acid entity, an aspartic acid (D)-amino acid entity, a glycine (G)-amino acid entity, a serine (S)-amino acid entity, a methionine (M)-amino acid entity, a cysteine (C)-amino acid entity, an ornithine (Orn)-amino acid entity, an arginine (R)-amino acid entity, a histidine (H)-amino acid entity, a tyrosine (Y)-amino acid entity, and a tryptophan (W)-amino acid entity.

In some embodiments, the disclosure is directed to a method of improving (e.g., increasing) myotube fusion index (fusion index) or myogenesis (e.g., in a cell, tissue, or a subject), the method comprising administering a composition comprising a glutamic acid (E)-amino acid entity, an aspartic acid (D)-amino acid entity, and a glycine (G)-amino acid entity.

In some embodiments, the disclosure is directed to a method of improving (e.g., increasing) myotube fusion index (fusion index) or myogenesis (e.g., in a cell, tissue, or a subject), the method comprising administering a composition comprising a leucine (L)-amino acid entity, an isoleucine (I)-amino acid entity, a valine (V)-amino acid entity, a glutamine (Q)-amino acid entity, a glycine (G)-amino acid entity, a serine (S)-amino acid entity, a methionine (M)-amino acid entity, a cysteine (C)-amino acid entity, an arginine (R)-amino acid entity, a histidine (H)-amino acid entity, a tyrosine (Y)-amino acid entity, and a tryptophan (W)-amino acid entity.

In some embodiments, the disclosure is directed to a method of improving (e.g., increasing) myotube fusion index (fusion index) or myogenesis (e.g., in a cell, tissue, or a subject), the method comprising administering a composition to comprising a leucine (L)-amino acid entity, an isoleucine (I)-amino acid entity, a valine (V)-amino acid entity, a glutamic acid (E)-amino acid entity, an aspartic acid (D)-amino acid entity, a serine (S)-amino acid entity, an ornithine (Orn)-amino acid entity, and a histidine (H)-amino acid entity.

In some embodiments, the disclosure is directed to a method of improving (e.g., increasing) myotube fusion index (fusion index) or myogenesis (e.g., in a cell, tissue, or a subject), the method comprising administering a composition to comprising a leucine (L)-amino acid entity, an isoleucine (I)-amino acid entity, a valine (V)-amino acid entity, a cysteine (C)-amino acid entity or a NAC entity, and a histidine (H)-amino acid entity.

### Dosage Regimens

The composition can be administered according to a dosage regimen described herein to, e.g., enhance muscle function in a subject (e.g., a human, such as a human with muscle atrophy). The composition can be administered according to a dosage regimen described herein to treat (e.g., inhibit, reduce, ameliorate, or prevent) a disorder, e.g., a muscle disease in a subject (e.g., a human). In some embodiments, the subject has a rare muscle disease. In some embodiments, the subject has muscle atrophy, sarcopenia, muscle deterioration, muscle decay, cachexia, drug-induced myopathy, muscular dystrophy, or myopenia. In some embodiments, the subject has a fracture or other trauma. In some embodiments, the subject has a statin-induced myopathy. In some embodiments, the subject has a steroid-induced myopathy. In some embodiments, the subject has an immunosuppressant-induced myopathy. In some embodiments, the subject has a chemotherapeutic-induced myopathy. In some embodiments, the subject has an alcohol-induced myopathy.

In some embodiments, the composition can be provided to a patient to enhance muscle function and/or treat a muscle disease or disorder (e.g., muscle atrophy, sarcopenia, muscle deterioration, muscle decay, cachexia, drug-induced myopathy, muscular dystrophy, or myopenia) in a patient in either a single or multiple dosage regimens. In some embodiments, doses can be administered, e.g., twice daily, three times daily, four times daily, five times daily, six times daily, seven times daily, or more. In some embodiments, the composition is administered for at least 2 days, 3 days, 4 days, 5 days, 6 days, 7 days, or 2 weeks. In some embodiments, the composition is administered for at least 10 weeks, 11 weeks, 12 weeks, 13 weeks, 14 weeks, 15 weeks, 16 weeks, 17 weeks, 18 weeks, 19 weeks, 20 weeks, or longer. In some embodiments, the composition can be administered chronically, e.g., more than 30 days, e.g., 31 days, 40 days. 50 days, 60 days, 3 months, 6 months, 9 months, one years two years, or three years).

In some embodiments, the composition is administered at a dose of about 4 g and about 80 g total amino acids, e.g., once per day, twice per day, three times per day, four times per day, five times per day, or six times per day (e.g., three times per day). In some embodiments, the composition is administered at a dose of about 5 g to about 15 g, about 10 g to about 20 g, about 20 g to about 40 g, or about 30 g to about 50 g total amino acids, e.g., once per day, twice per day, three times per day, four times per day, five times per day, or six times per day (e.g., three times per day).

In some embodiments, the composition is administered at a dose of about 5 g to about 15 g (e.g., about 6 g total amino acids), e.g., once per day, twice per day, three times per day, four times per day, five times per day, or six times per day (e.g., three times per day). In an embodiment, about 18 g total amino acids is administered per day to enhance muscle function in a subject (e.g., the subject has or is identified as having decreased muscle function due to aging, injury, atrophy, infection, or disease).

In some embodiments, the composition is administered at a dose of about 5 g to about 15 g (e.g., about 6 g total amino acids), e.g., once per day, twice per day, three times per day, four times per day, five times per day, or six times per day (e.g., three times per day). In an embodiment, about 18 g total amino acids is administered per day to treat a muscle disease or disorder (e.g., muscle atrophy, sarcopenia, muscle deterioration, muscle decay, cachexia, drug-induced myopathy, muscular dystrophy, or myopenia) in a subject. In an embodiment, about 23 g total amino acids is administered per day to treat a muscle disease or disorder (e.g., muscle atrophy, sarcopenia, muscle deterioration, muscle decay, cachexia, drug-induced myopathy, muscular dystrophy, or myopenia) in a subject. In an embodiment, about 48 g total amino acids is administered per day to treat a muscle disease or disorder (e.g., muscle atrophy, sarcopenia, muscle deterioration, muscle decay, cachexia, drug-induced myopathy, muscular dystrophy, or myopenia) in a subject. In an embodiment, about 68 g total amino acids is administered per day to treat a muscle disease or disorder (e.g., muscle atrophy, sarcopenia, muscle deterioration, muscle decay, cachexia, drug-induced myopathy, muscular dystrophy, or myopenia) in a subject. In an embodiment, about 72 g total amino acids is administered per day to treat a muscle disease or disorder (e.g., muscle atrophy, sarcopenia, muscle deterioration, muscle decay, cachexia, drug-induced myopathy, muscular dystrophy, or myopenia) in a subject.

In some embodiments, the composition is administered at a dose of about 15 g to about 40 g (e.g., about 24 g total amino acids), e.g., once per day, twice per day, three times per day, four times per day, five times per day, or six times per day (e.g., three times per day). Thus, about 68 g or about 72 g total amino acids is administered per day to enhance muscle function in a subject (e.g., the subject has or is identified as having decreased muscle function due to aging, injury, atrophy, infection, or disease).

In some embodiments, the composition is administered at a dose of about 15 g to about 40 g (e.g., about 24 g total amino acids), e.g., once per day, twice per day, three times per day, four times per day, five times per day, or six times per day (e.g., three times per day). Thus, about 68 g or about 72 g total amino acids is administered per day to treat a muscle disease or disorder (e.g., muscle atrophy, sarcopenia, muscle deterioration, muscle decay, cachexia, drug-induced myopathy, muscular dystrophy, or myopenia) in a subject.

In some embodiments, the composition is administered every 2 hours, every 3 hours, every 4 hours, every 5 hours, every 6 hours, every 7 hours, every 8 hours, every 9 hours, or every 10 hours to enhance muscle function in a subject (e.g., the subject has or is identified as having decreased muscle function due to aging, injury, atrophy, infection, or disease).

In some embodiments, the composition is administered prior to a meal (e.g., one, two, or more (e.g., all) of breakfast, lunch, or dinner). In some embodiments, the composition is administered concurrent with a meal (e.g., one, two, or more (e.g., all) of breakfast, lunch, or dinner). In some embodiments, the composition is administered following a meal (e.g., one, two, or more (e.g., all) of breakfast, lunch, or dinner).

### Dietary Compositions

The composition including amino acid entities can be a dietary composition, e.g., chosen from a medical food, a functional food, or a supplement.

The composition including amino acid entities can be for use as a dietary composition, e.g., chosen from a medical food, a functional food, or a supplement. In some embodiments, the dietary composition is for use in a method comprising administering the composition to a subject.

In some embodiments, the composition is for use in treating a subject having or identified as having decreased muscle function due to aging, injury, atrophy, infection, or disease.

In some embodiments, the subject has or is identified as having muscle deterioration, muscle decay, muscle atrophy, cachexia, sarcopenia, steroid myopathy, or muscular dystrophy

In some embodiments, the subject has one or both of type 2 diabetes or a relatively high BMI.

In some embodiments, the composition promotes weight loss in the subject.

In some embodiments, administration of the dietary composition results in an improvement in one or more metabolic symptoms in the subject, e.g., one or more metabolic symptoms is selected from the following: increased free fatty acid and lipid metabolism, improved mitochondrial function, white adipose tissue (WAT) browning, decreased reactive oxygen species (ROS), increased levels of glutathione (GSH), decreased hepatic inflammation, decreased hepatocyte ballooning, improved gut barrier function, increased insulin secretion, or glucose tolerance. In certain embodiments, administration of the composition results in an improvement in one or more metabolic symptoms after a treatment period of 24 hours.

### Methods of Providing an Amino Acid to a Subject

The present disclosure features a method of providing amino acid entities to a subject comprising administering to the subject an effective amount of a composition described herein. In some embodiments, at least one amino acid entity is not a peptide of more than 20 amino acid residues in length.

The present disclosure also features a method of increasing one, two, three, or more (e.g., all) amino acid entities in a subject comprising administering to the subject an effective amount of the composition described herein. In some embodiments, administration of the composition results in an increase in the amino acid entities in one, two, three, or more (e.g., all) of blood, plasma, or serum of the subject, e.g., in a blood, plasma, or serum sample from the subject.

### Biomarkers

Any of the methods disclosed herein can include evaluating or monitoring the effectiveness of administering a composition described herein to a subject. In some embodiments, the subject is in need of muscle function enhancement (e.g., a subject having muscle deterioration, muscle decay, muscle atrophy, cachexia, sarcopenia, drug-induced myopathy, muscular dystrophy, or myopenia).

In some embodiments, the value of effectiveness to the composition in treating a subject comprises a measure of the levels of one, two, three, four, five, six, seven, eight, nine, ten, eleven, twelve, thirteen, fourteen, fifteen, or more (e.g., all) of the following:
a) myostatin;
b) myoglobin;
c) Cortisol-AM;
d) C-reactive protein;
e) insulin;
f) cytokines (e.g., one, two, three, four, five, six, or more (e.g., all of IL-1A RBM, IL-1RA, IL-1 RI, IL-1 RII, IL-12, IL-18, or MCP-1);
g) GDF-11;
h) P3NP;
i) IGF-1;
j) IGFBP1;
k) IGFBP3;
l) FGF21;
m) DHEAS;
n) mTORC1;
o) Gcn2; or
p) AMP-activated protein kinase (AMPK).

In some embodiments of any of the methods disclosed herein, the measure of one or more of a)-p) is obtained from a sample acquired from the subject, e.g., a subject in need of muscle function enhancement (e.g., a subject having muscle deterioration, muscle decay, muscle atrophy, cachexia, sarcopenia, drug-induced myopathy, muscular dystrophy, or myopenia). In some embodiments, the sample is chosen from a blood sample (e.g., a plasma sample) or a muscle sample.

In some embodiments, the subject is evaluated prior to receiving, during, or after receiving, the composition.

In some embodiments, administration of the composition (e.g., at a dose of about 4 g to about 80 g total amino acids, e.g., about 6 g, about 12 g, about 18 g, or about 24 g three times daily), results in an improvement of one, two, three, four, five, six, seven, eight, nine, ten, eleven, twelve, thirteen, fourteen, fifteen, or more (e.g., all) of the following:
a) myostatin;
b) myoglobin;
c) Cortisol-AM;
d) C-reactive protein;
e) insulin;
f) cytokines (e.g., one, two, three, four, five, six, or more (e.g., all of IL-1A RBM, IL-1RA, IL-1 RI, IL-1 RII, IL-12, IL-18, or MCP-1);
g) GDF-11;
h) P3NP;
i) IGF-1;
j) IGFBP1;
k) IGFBP3;
l) FGF21;
m) DHEAS;
n) mTORC1;
o) Gcn2; or
p) AMP-activated protein kinase (AMPK).

In some embodiments, administration of the composition to the subject results in a decrease in levels of one, two, three, four, five, six, or more (e.g., all) of myoglobin, myostatin, GDF-11, cortisol-AM, C-reactive protein, insulin, or cytokines (e.g., one, two, three, four, five, six, or more (e.g., all of IL-1A RBM, IL-1RA, IL-1 RI, IL-1 RII, IL-12, IL-18, or MCP-1) in the subject (Table 4). In some embodiments, administration of the composition to the subject results in an increase in levels of one, two, three, four, five, six, or more (e.g., all) of P3NP, IGF-1, IGFBP1, IGFBP3, FGF-21, DHEAS, or mTORC1 in the subject (Table 3).

TABLE 3
Biomarkers to determine effect of the composition on muscle biology.

| | | Expected | |
|---|---|---|---|
| | | Change in | |
| | | Response | |
| | | to | Additional information |
| Bio- | | Compo- | regarding biomarker change on |
| marker | Category | sition | muscle synthesis and/or breakdown |
| Myo- | Muscle | Down | Decrease suggests a reduction in |
| globin | biology | | muscle breakdown and autophagy |
| Myostatin | Muscle | Down | Myostatin acts to inhibit muscle |
| | | | synthesis—decrease in levels |
| | biology | | indicate increase anabolism |
| | | | and increased myogenesis |
| IL-6, | Muscle | Up | Myokines produced by muscle |
| FGF21, | Biology | | in response to physiological |
| Irisin | | | stress such as exercise or |
| | | | fasting. Expected increase levels: |
| | | | IL6—will improve myogenesis |
| | | | FGF21—improve whole body |
| | | | metabolism (eg; |
| | | | diminished fat stores) |
| | | | Irisin—Induces WAT browing and |
| | | | improved energy expenditure |
| P3NP | Muscle | Up | P3NP is released during collagen |
| | biology | | synthesis in muscle |
| | | | Increased circulating P3NP |
| | | | indicates muscle growth, muscle |
| | | | repair and fibrosis |
| Cortisol- | Endo- | Down | Hepatic and neuroendocrine |
| AM | crine | | signals of acute and chronic |
| | | | inflammation which are associated |
| | | | with anabolic resistance |
| C-reactive | Endo- | Down | Expected to decrease with improved |
| protein | crine | | protein synthesis and muscle growth |
| IGF-1, | Endo- | Up | Endocrine molecules involved |
| IGFBP1, | crine | | in regulating protein synthesis |
| IGFBP3, | | | and myogenesis as stimulators/ |
| FGF21, | | | potentiators or inhibitors |
| DHEAS | | | Increase in potentiator levels and |
| | | | decrease in inhibitor levels are |
| | | | supportive of net anabolism |
| Insulin | Endo- | Down | Decrease indicates moderation in |
| | crine | | insulin resistance, and |
| | (glucose | | increased glucose handling |
| | tolerance) | | and anabolic sensitivity |
| | | | Increased muscle wasting is |
| | | | associated with a strong |
| | | | inflammatory response |
| IL1ARBM, | Inflam- | Down | Reduced levels of these inflam- |
| IL1RA, | mation | | mation biomarkers indicate |
| IL1RI, | | | reduction in inflammation |
| IL1RII, | | | Overall profile of these biomarker |
| IL-12, | | | can further provide dynamic |
| IL-18, | | | assessment on interleukin |
| MCP-1, | | | response to the composition |
| cytokines | | | |

In some embodiments, administration of the composition (e.g., at a dose of about 4 g and about 80 g total amino acids, e.g., about 6 g, about 12 g, about 18 g, or about 24 g three times daily), results in an improvement in one, two, three, four, five, or more (e.g., all) of a)-p) after a treatment period of, about 24 hours, about 72 hours, about 1 week, about 2 weeks, about 3 weeks, about 4 weeks, about 5 weeks, about 6 weeks, about 7 weeks, about 8 weeks, about 9 weeks, about 10 weeks, about 11 weeks, or 12 weeks. In certain embodiments, administration of the composition results in an improvement in one, two, three, four, five, six, seven, eight, nine, ten, eleven, twelve, thirteen, fourteen, fifteen, or more (e.g., all) of a)-p) after a treatment period of about 2 weeks.

### NUMBERED EMBODIMENTS

The invention is further described with reference to the following numbered embodiments.

1. A composition comprising:

a) a leucine (L)-amino acid entity;

b) an isoleucine (I)-amino acid entity;

c) a valine (V)-amino acid entity;

d) an aspartic acid (D)-amino acid entity;

e) a methionine (M)-amino acid entity;

f) a cysteine (C)-amino acid entity;

g) an arginine (R)-amino acid entity;

h) a histidine (H)-amino acid entity;

i) a tyrosine (Y)-amino acid entity; and

j) a tryptophan (W)-amino acid entity;

provided that:

(i) at least one amino acid entity is not provided as a peptide of more than 20 amino acid residues in length;

(ii) the amino acid entities of (a)-(j) are selected from Table 2; and

(iii) the composition comprises fewer than 20 different amino acid entities.

2. The composition of claim 1, wherein:

a) the leucine (L)-amino acid entity is L-leucine or a salt thereof;

b) the isoleucine (I)-amino acid entity is L-isoleucine or a salt thereof;

c) the valine (V)-amino acid entity is L-valine or a salt thereof;

d) the aspartic acid (D)-amino acid entity is L-aspartate or a salt thereof;

e) the methionine (M)-amino acid entity is L-methionine or a salt thereof;

f) the cysteine (C)-amino acid entity is N-acetylcysteine or a salt thereof;

g) the arginine (R)-amino acid entity is L-arginine or a salt thereof;

h) the histidine (H)-amino acid entity is L-histidine or a salt thereof;

i) the tyrosine (Y)-amino acid entity is L-tyrosine or a salt thereof; and

j) the tryptophan (W)-amino acid entity is L-tryptophan or a salt thereof.

3. The composition of embodiment 1, wherein the total wt. % of (a)-(j) is greater than the total wt. % of other amino acid entities in the composition.
4. A composition comprising:

a) a leucine (L)-amino acid entity;

b) an isoleucine (I)-amino acid entity;

c) a valine (V)-amino acid entity;

d) a glutamine (Q)-amino acid entity;

e) a glutamic acid (E)-amino acid entity;

f) an aspartic acid (D)-amino acid entity;

g) a glycine (G)-amino acid entity;

h) a serine (S)-amino acid entity;

i) a methionine (M)-amino acid entity;

j) a cysteine (C)-amino acid entity;

k) an ornithine (Orn)-amino acid entity;

l) an arginine (R)-amino acid entity;

m) a histidine (H)-amino acid entity;

n) a tyrosine (Y)-amino acid entity; and

o) a tryptophan (W)-amino acid entity;

provided that:

(i) at least one amino acid entity is not provided as a peptide of more than 20 amino acid residues in length;

(ii) the amino acid entities of (a)-(o) are selected from Table 2; and

(iii) the composition comprises fewer than 20 different amino acid entities.

5. The composition of embodiment 4, wherein comprising:

a) the leucine (L)-amino acid entity is L-leucine or a salt thereof;

b) the isoleucine (I)-amino acid entity is L-isoleucine or a salt thereof;

c) the valine (V)-amino acid entity is L-valine or a salt thereof;

d) the glutamine (Q)-amino acid entity is L-glutamine or a salt thereof;

e) the glutamic acid (E)-amino acid entity is L-glutamate or a salt thereof;

f) the aspartic acid (D)-amino acid entity is L-aspartate or a salt thereof;

g) the glycine (G)-amino acid entity is Glycine or a salt thereof;

h) the serine (S)-amino acid entity is L-serine or a salt thereof;

i) the methionine (M)-amino acid entity is L-methionine or a salt thereof;

j) the cysteine (C)-amino acid entity is N-acetylcysteine or a salt thereof;

k) the ornithine (Orn)-amino acid entity is L-ornithine or a salt thereof;

l) the arginine (R)-amino acid entity is L-arginine or a salt thereof;

m) the histidine (H)-amino acid entity is L-histidine or a salt thereof;

n) the tyrosine (Y)-amino acid entity is L-tyrosine or a salt thereof; and

o) the tryptophan (W)-amino acid entity is L-tryptophan or a salt thereof.

6. The composition of embodiment 4, wherein the total wt. % of (a)-(o) is greater than the total wt. % of other amino acid entities in the composition.
7. A composition comprising:

a) a leucine (L)-amino acid entity;

b) an isoleucine (I)-amino acid entity;

c) a valine (V)-amino acid entity;

d) a glutamic acid (E)-amino acid entity;

e) an aspartic acid (D)-amino acid entity;

f) a serine (S)-amino acid entity;

g) a cysteine (C)-amino acid entity; and

h) a histidine (H)-amino acid entity;

provided that:

(i) at least one amino acid entity is not provided as a peptide of more than 20 amino acid residues in length;

(ii) the amino acid entities of (a)-(h) are selected from Table 2; and

(iii) the composition comprises fewer than 20 different amino acid entities.

8. The composition of embodiment 7, wherein:

a) the leucine (L)-amino acid entity is L-leucine or a salt thereof;

b) the isoleucine (I)-amino acid entity is L-isoleucine or a salt thereof;

c) the valine (V)-amino acid entity is L-valine or a salt thereof;

d) the glutamic acid (E)-amino acid entity is L-glutamate or a salt thereof;

e) the aspartic acid (D)-amino acid entity is L-aspartate or a salt thereof;

f) the serine (S)-amino acid entity is L-serine or a salt thereof;

g) the cysteine (C)-amino acid entity is N-acetylcysteine or a salt thereof; and

h) the histidine (H)-amino acid entity is L-histidine or a salt thereof.

9. The composition of embodiment 7, wherein the total wt. % of (a)-(h) is greater than the total wt. % of other amino acid entities in the composition.
10. A composition comprising:

a) a leucine (L)-amino acid entity;

b) an isoleucine (I)-amino acid entity;

c) a valine (V)-amino acid entity;

d) a methionine (M)-amino acid entity;

e) a cysteine (C)-amino acid entity;

f) an arginine (R)-amino acid entity;

g) a histidine (H)-amino acid entity;

h) a tyrosine (Y)-amino acid entity; and

i) a tryptophan (W)-amino acid entity;

(i) at least one amino acid entity is not provided as a peptide of more than 20 amino acid residues in length;

(ii) the amino acid entities of (a)-(i) are selected from Table 2; and

(iii) the composition comprises fewer than 20 different amino acid entities.

11. The composition of embodiment 10, wherein:

a) the leucine (L)-amino acid entity is L-leucine or a salt thereof;

b) the isoleucine (I)-amino acid entity is L-isoleucine or a salt thereof;

c) the valine (V)-amino acid entity is L-valine or a salt thereof;

d) the methionine (M)-amino acid entity is L-methionine or a salt thereof;

e) the cysteine (C)-amino acid entity is N-acetylcysteine or a salt thereof;

f) the arginine (R)-amino acid entity is L-arginine or a salt thereof;

g) the histidine (H)-amino acid entity is L-histidine or a salt thereof;

h) the tyrosine (Y)-amino acid entity is L-tyrosine or a salt thereof; and

i) the tryptophan (W)-amino acid entity is L-tryptophan or a salt thereof.

12. The composition of embodiment 10, wherein the total wt. % of (a)-(i) is greater than the total wt. % of other amino acid entities in the composition.
13. A composition comprising:

a) a leucine (L)-amino acid entity;

b) an isoleucine (I)-amino acid entity;

c) a valine (V)-amino acid entity;

d) a glutamic acid (E)-amino acid entity;

e) an aspartic acid (D)-amino acid entity;

f) a serine (S)-amino acid entity;

g) a cysteine (C)-amino acid entity;

h) an ornithine (Orn)-amino acid entity; and

i) a histidine (H)-amino acid entity;

provided that:

(i) at least one amino acid entity is not provided as a peptide of more than 20 amino acid residues in length;

(ii) the amino acid entities of (a)-(i) are selected from Table 2; and

(iii) the composition comprises fewer than 20 different amino acid entities.

14. The composition of embodiment 13, wherein:

a) the leucine (L)-amino acid entity is L-leucine or a salt thereof;

b) the isoleucine (I)-amino acid entity is L-isoleucine or a salt thereof;

c) the valine (V)-amino acid entity is L-valine or a salt thereof;

d) the glutamic acid (E)-amino acid entity is L-glutamate or a salt thereof;

e) the aspartic acid (D)-amino acid entity is L-aspartate or a salt thereof;

f) the serine (S)-amino acid entity is L-serine or a salt thereof;

g) the cysteine (C)-amino acid entity is N-acetylcysteine or a salt thereof;

h) the ornithine (Orn)-amino acid entity is L-ornithine or a salt thereof; and

i) the histidine (H)-amino acid entity is L-histidine or a salt thereof.

15. The composition of embodiment 13, wherein the total wt. % of (a)-(i) is greater than the total wt. % of other amino acid entities in the composition.
16. A composition comprising:

a) a leucine (L)-amino acid entity;

b) an isoleucine (I)-amino acid entity;

c) a valine (V)-amino acid entity;

d) an aspartic acid (D)-amino acid entity;

e) a cysteine (C)-amino acid entity; and

f) an ornithine (Orn)-amino acid entity;

provided that:

(i) at least one amino acid entity is not provided as a peptide of more than 20 amino acid residues in length;

(ii) the amino acid entities of (a)-(f) are selected from Table 2; and

(iii) the composition comprises fewer than 20 different amino acid entities.

17. The composition of embodiment 16, wherein:

a) the leucine (L)-amino acid entity is L-leucine or a salt thereof;

b) the isoleucine (I)-amino acid entity is L-isoleucine or a salt thereof;

c) the valine (V)-amino acid entity is L-valine or a salt thereof;

d) the aspartic acid (D)-amino acid entity is L-aspartate or a salt thereof;

e) the cysteine (C)-amino acid entity is N-acetylcysteine or a salt thereof; and

f) the ornithine (Orn)-amino acid entity is L-ornithine or a salt thereof.

18. The composition of embodiment 16, wherein the total wt. % of (a)-(f) is greater than the total wt. % of other amino acid entities in the composition.
19. A composition comprising:

a) a leucine (L)-amino acid entity;

b) an isoleucine (I)-amino acid entity;

c) a valine (V)-amino acid entity;

d) a glutamic acid (E)-amino acid entity;

e) an aspartic acid (D)-amino acid entity;

f) a serine (S)-amino acid entity;

g) a cysteine (C)-amino acid entity;

h) an ornithine (Orn)-amino acid entity;

i) a histidine (H)-amino acid entity;

j) a tyrosine (Y)-amino acid entity; and

k) a tryptophan (W)-amino acid entity;

provided that:

(i) at least one amino acid entity is not provided as a peptide of more than 20 amino acid residues in length;

(ii) the amino acid entities of (a)-(k) are selected from Table 2; and

(iii) the composition comprises fewer than 20 different amino acid entities.

20. The composition of embodiment 19, wherein:

a) the leucine (L)-amino acid entity is L-leucine or a salt thereof;

b) the isoleucine (I)-amino acid entity is L-isoleucine or a salt thereof;

c) the valine (V)-amino acid entity is L-valine or a salt thereof;

d) the glutamic acid (E)-amino acid entity is L-glutamate or a salt thereof;

e) the aspartic acid (D)-amino acid entity is L-aspartate or a salt thereof;

f) the serine (S)-amino acid entity is L-serine or a salt thereof;

g) the cysteine (C)-amino acid entity is N-acetylcysteine or a salt thereof;

h) the ornithine (Orn)-amino acid entity is L-ornithine or a salt thereof;

i) the histidine (H)-amino acid entity is L-histidine or a salt thereof;

j) the tyrosine (Y)-amino acid entity is L-tyrosine or a salt thereof; and

k) the tryptophan (W)-amino acid entity is L-tryptophan or a salt thereof.

21. The composition of embodiment 19, wherein the total wt. % of (a)-(k) is greater than the total wt. % of other amino acid entities in the composition.
22. A composition comprising:

a) a leucine (L)-amino acid entity;

b) an isoleucine (I)-amino acid entity;

c) a valine (V)-amino acid entity;

d) a glutamine (Q)-amino acid entity;

e) a glycine (G)-amino acid entity;

f) a serine (S)-amino acid entity;

g) a methionine (M)-amino acid entity;

h) a cysteine (C)-amino acid entity;

i) an arginine (R)-amino acid entity;

j) a histidine (H)-amino acid entity;

k) a tyrosine (Y)-amino acid entity; and

l) a tryptophan (W)-amino acid entity;

provided that:

(i) at least one amino acid entity is not provided as a peptide of more than 20 amino acid residues in length;

(ii) the amino acid entities of (a)-(l) are selected from Table 2; and

(iii) the composition comprises fewer than 20 different amino acid entities.

23. The composition of embodiment 22, wherein:

a) the leucine (L)-amino acid entity is L-leucine or a salt thereof;

b) the isoleucine (I)-amino acid entity is L-isoleucine or a salt thereof;

c) the valine (V)-amino acid entity is L-valine or a salt thereof;

d) the glutamine (Q)-amino acid entity is L-glutamine or a salt thereof;

e) the glycine (G)-amino acid entity is glycine or a salt thereof;

f) the serine (S)-amino acid entity is L-serine or a salt thereof;

g) the methionine (M)-amino acid entity is L-methionine or a salt thereof;

h) the cysteine (C)-amino acid entity is N-acetylcysteine or a salt thereof;

i) the arginine (R)-amino acid entity is L-arginine or a salt thereof;

j) the histidine (H)-amino acid entity is L-histidine or a salt thereof;

k) the tyrosine (Y)-amino acid entity is L-tyrosine or a salt thereof; and

l) the tryptophan (W)-amino acid entity is L-tryptophan or a salt thereof.

24. The composition of embodiment 22, wherein the total wt. % of (a)-(l) is greater than the total wt. % of other amino acid entities in the composition.
25. A composition comprising:

a) a glutamine (Q)-amino acid entity;

b) a glutamic acid (E)-amino acid entity;

c) an aspartic acid (D)-amino acid entity;

d) a glycine (G)-amino acid entity; and

e) a serine (S)-amino acid entity;

provided that:

(i) at least one amino acid entity is not provided as a peptide of more than 20 amino acid residues in length;

(ii) the amino acid entities of (a)-(e) are selected from Table 2; and

(iii) the composition comprises fewer than 20 different amino acid entities.

26. The composition of embodiment 25, wherein:

a) the glutamine (Q)-amino acid entity is L-glutamine or a salt thereof;

b) the glutamic acid (E)-amino acid entity is L-glutamate or a salt thereof;

c) the aspartic acid (D)-amino acid entity is L-aspartate or a salt thereof;

d) the glycine (G)-amino acid entity is glycine or a salt thereof; and

e) the serine (S)-amino acid entity is L-serine or a salt thereof.

27. The composition of embodiment 25, wherein the total wt. % of (a)-(e) is greater than the total wt. % of other amino acid entities in the composition.
28. A composition comprising:

a) a leucine (L)-amino acid entity;

b) an isoleucine (I)-amino acid entity;

c) a valine (V)-amino acid entity;

d) an ornithine (Orn)-amino acid entity; and

e) an arginine (R)-amino acid entity;

provided that:

(i) at least one amino acid entity is not provided as a peptide of more than 20 amino acid residues in length;

(ii) the amino acid entities of (a)-(e) are selected from Table 2; and

(iii) the composition comprises fewer than 20 different amino acid entities.

29. The composition of embodiment 28, wherein:

a) the leucine (L)-amino acid entity is L-leucine or a salt thereof;

b) the isoleucine (I)-amino acid entity is L-isoleucine or a salt thereof;

c) the valine (V)-amino acid entity is L-valine or a salt thereof;

d) the ornithine (Orn)-amino acid entity is L-ornithine or a salt thereof; and

e) the arginine (R)-amino acid entity is L-arginine or a salt thereof.

30. The composition of embodiment 28, wherein the total wt. % of (a)-(e) is greater than the total wt. % of other amino acid entities in the composition.
31. A composition comprising:

a) a leucine (L)-amino acid entity;

b) an isoleucine (I)-amino acid entity;

c) a valine (V)-amino acid entity;

d) a glutamic acid (E)-amino acid entity;

e) an aspartic acid (D)-amino acid entity;

f) a serine (S)-amino acid entity;

g) an ornithine (Orn)-amino acid entity; and

h) a histidine (H)-amino acid entity;

provided that:

(i) at least one amino acid entity is not provided as a peptide of more than 20 amino acid residues in length;

(ii) the amino acid entities of (a)-(h) are selected from Table 2; and

(iii) the composition comprises fewer than 20 different amino acid entities.

32. The composition of embodiment 31, wherein:

a) the leucine (L)-amino acid entity is L-leucine or a salt thereof;

b) the isoleucine (I)-amino acid entity is L-isoleucine or a salt thereof;

c) the valine (V)-amino acid entity is L-valine or a salt thereof;

d) the glutamic acid (E)-amino acid entity is L-glutamate or a salt thereof;

e) the aspartic acid (D)-amino acid entity is L-aspartate or a salt thereof;

f) the serine (S)-amino acid entity is L-serine or a salt thereof;

g) the ornithine (Orn)-amino acid entity is L-ornithine or a salt thereof; and

h) the histidine (H)-amino acid entity is L-histidine or a salt thereof.

33. The composition of embodiment 31, wherein the total wt. % of (a)-(h) is greater than the total wt. % of other amino acid entities in the composition.
34. A composition comprising:

a) a leucine (L)-amino acid entity;

b) an isoleucine (I)-amino acid entity;

c) a valine (V)-amino acid entity;

d) a methionine (M)-amino acid entity; and

e) a cysteine (C)-amino acid entity;

provided that:

(i) at least one amino acid entity is not provided as a peptide of more than 20 amino acid residues in length;

(ii) the amino acid entities of (a)-(e) are selected from Table 2; and

(iii) the composition comprises fewer than 20 different amino acid entities.

35. The composition of embodiment 34, wherein:

a) the leucine (L)-amino acid entity is L-leucine or a salt thereof;

b) the isoleucine (I)-amino acid entity is L-isoleucine or a salt thereof;

c) the valine (V)-amino acid entity is L-valine or a salt thereof;

d) the methionine (M)-amino acid entity is L-methionine or a salt thereof; and

e) the cysteine (C)-amino acid entity is N-acetylcysteine or a salt thereof.

36. The composition of embodiment 34, wherein the total wt. % of (a)-(e) is greater than the total wt. % of other amino acid entities in the composition.
37. A composition comprising:

a) a leucine (L)-amino acid entity;

b) an isoleucine (I)-amino acid entity;

c) a valine (V)-amino acid entity;

d) a glutamine (Q)-amino acid entity;

e) a glutamic acid (E)-amino acid entity;

f) an aspartic acid (D)-amino acid entity;

g) a glycine (G)-amino acid entity;

h) a serine (S)-amino acid entity;

i) a cysteine (C)-amino acid entity; and

j) an ornithine (Orn)-amino acid entity;

provided that:

(i) at least one amino acid entity is not provided as a peptide of more than 20 amino acid residues in length;

(ii) the amino acid entities of (a)-(j) are selected from Table 2; and

(iii) the composition comprises fewer than 20 different amino acid entities.

38. The composition of embodiment 37, wherein:

a) the leucine (L)-amino acid entity is L-leucine or a salt thereof;

b) the isoleucine (I)-amino acid entity is L-isoleucine or a salt thereof;

c) the valine (V)-amino acid entity is L-valine or a salt thereof;

d) the glutamine (Q)-amino acid entity is L-glutamine or a salt thereof;

e) the glutamic acid (E)-amino acid entity is L-glutamate or a salt thereof;

f) the aspartic acid (D)-amino acid entity is L-aspartate or a salt thereof;

g) the glycine (G)-amino acid entity is glycine or a salt thereof;

h) the serine (S)-amino acid entity is L-serine or a salt thereof;

i) the cysteine (C)-amino acid entity is N-acetylcysteine or a salt thereof; and

j) the ornithine (Orn)-amino acid entity is L-ornithine or a salt thereof.

39. The composition of embodiment 37, wherein the total wt. % of (a)-(j) is greater than the total wt. % of other amino acid entities in the composition.
40. A composition comprising:

a) a glutamine (Q)-amino acid entity;

b) a glutamic acid (E)-amino acid entity;

c) an aspartic acid (D)-amino acid entity;

d) a glycine (G)-amino acid entity;

e) an ornithine (Orn)-amino acid entity; and

f) an arginine (R)-amino acid entity;

provided that:

(i) at least one amino acid entity is not provided as a peptide of more than 20 amino acid residues in length;

(ii) the amino acid entities of (a)-(f) are selected from Table 2; and

(iii) the composition comprises fewer than 20 different amino acid entities.

41. The composition of embodiment 40, wherein:

a) the glutamine (Q)-amino acid entity is L-glutamine or a salt thereof;

b) the glutamic acid (E)-amino acid entity is L-glutamate or a salt thereof;

c) the aspartic acid (D)-amino acid entity is L-aspartate or a salt thereof;

d) the glycine (G)-amino acid entity is glycine or a salt thereof;

e) the ornithine (Orn)-amino acid entity is L-ornithine or a salt thereof; and

f) the arginine (R)-amino acid entity is L-arginine or a salt thereof.

42. The composition of embodiment 40, wherein the total wt. % of (a)-(f) is greater than the total wt. % of other amino acid entities in the composition.
43. A composition comprising:

a) a leucine (L)-amino acid entity;

b) an isoleucine (I)-amino acid entity;

c) a valine (V)-amino acid entity;

d) a cysteine (C)-amino acid entity; and

e) a histidine (H)-amino acid entity;

provided that:

(i) at least one amino acid entity is not provided as a peptide of more than 20 amino acid residues in length;

(ii) the amino acid entities of (a)-(e) are selected from Table 2; and

(iii) the composition comprises fewer than 20 different amino acid entities.

44. The composition of embodiment 43, wherein:

a) the leucine (L)-amino acid entity is L-leucine or a salt thereof;

b) the isoleucine (I)-amino acid entity is L-isoleucine or a salt thereof;

c) the valine (V)-amino acid entity is L-valine or a salt thereof;

d) the cysteine (C)-amino acid entity is N-acetylcysteine or a salt thereof; and

e) the histidine (H)-amino acid entity is L-histidine or a salt thereof.

45. The composition of embodiment 43, wherein the total wt. % of (a)-(e) is greater than the total wt. % of other amino acid entities in the composition.
46. A composition comprising:

a) a leucine (L)-amino acid entity;

b) an isoleucine (I)-amino acid entity;

c) a valine (V)-amino acid entity;

d) a glutamic acid (E)-amino acid entity;

e) an aspartic acid (D)-amino acid entity; and

f) a glycine (G)-amino acid entity;

provided that:

(i) at least one amino acid entity is not provided as a peptide of more than 20 amino acid residues in length;

(ii) the amino acid entities of (a)-(f) are selected from Table 2; and

(iii) the composition comprises fewer than 20 different amino acid entities.

47. The composition of embodiment 46, wherein:

a) the leucine (L)-amino acid entity is L-leucine or a salt thereof;

b) the isoleucine (I)-amino acid entity is L-isoleucine or a salt thereof;

c) the valine (V)-amino acid entity is L-valine or a salt thereof;

d) the glutamic acid (E)-amino acid entity is L-glutamate or a salt thereof;

e) the aspartic acid (D)-amino acid entity is L-aspartate or a salt thereof; and

f) the glycine (G)-amino acid entity is glycine or a salt thereof.

48. The composition of embodiment 46, wherein the total wt. % of (a)-(f) is greater than the total wt. % of other amino acid entities in the composition.
49. A composition comprising:

a) a leucine (L)-amino acid entity;

b) an isoleucine (I)-amino acid entity;

c) a valine (V)-amino acid entity;

d) a cysteine (C)-amino acid entity; and

e) an ornithine (Orn)-amino acid entity;

provided that:

(i) at least one amino acid entity is not provided as a peptide of more than 20 amino acid residues in length;

(ii) the amino acid entities of (a)-(e) are selected from Table 2; and

(iii) the composition comprises fewer than 20 different amino acid entities.

50. The composition of embodiment 49, wherein:

a) the leucine (L)-amino acid entity is L-leucine or a salt thereof;

b) the isoleucine (I)-amino acid entity is L-isoleucine or a salt thereof;

c) the valine (V)-amino acid entity is L-valine or a salt thereof;

d) the cysteine (C)-amino acid entity or NAC entity is N-acetylcysteine; and

e) the ornithine (Orn)-amino acid entity is ornithine or a salt thereof.

51. The composition of embodiment 49, wherein the total wt. % of (a)-(e) is greater than the total wt. % of other amino acid entities in the composition.
52. A composition comprising:

a) a glutamic acid (E)-amino acid entity;

b) an aspartic acid (D)-amino acid entity; and

c) a glycine (G)-amino acid entity;

provided that:

(i) at least one amino acid entity is not provided as a peptide of more than 20 amino acid residues in length;

(ii) the amino acid entities of (a)-(c) are selected from Table 2; and

(iii) the composition comprises fewer than 20 different amino acid entities.

53. The composition of embodiment 52, wherein:

a) the glutamic acid (E)-amino acid entity is L-glutamate or a salt thereof;

b) the aspartic acid (D)-amino acid entity is L-aspartate or a salt thereof; and

c) the glycine (G)-amino acid entity is glycine or a salt thereof.

54. The composition of embodiment 52, wherein the total wt. % of (a)-(c) is greater than the total wt. % of other amino acid entities in the composition.
55. A composition comprising:

a) a glutamine (Q)-amino acid entity; and

b) a glutamic acid (E)-amino acid entity;

provided that:

(i) at least one amino acid entity is not provided as a peptide of more than 20 amino acid residues in length;

(ii) the amino acid entities of (a)-(b) are selected from Table 2; and

(iii) the composition comprises fewer than 20 different amino acid entities.

56. The composition of embodiment 55, wherein:

a) the glutamine (Q)-amino acid entity is L-glutamine or a salt thereof; and

b) the glutamic acid (E)-amino acid entity is L-glutamic acid or a salt thereof.

57. The composition of embodiment 55, wherein the total wt. % of (a)-(b) is greater than the total wt. % of other amino acid entities in the composition.
58. A composition comprising:

a) a methionine (M)-amino acid entity; and

b) a cysteine (C)-amino acid entity;

provided that:

(i) at least one amino acid entity is not provided as a peptide of more than 20 amino acid residues in length;

(ii) the amino acid entities of (a)-(b) are selected from Table 2; and

(iii) the composition comprises fewer than 20 different amino acid entities.

59. The composition of embodiment 58, wherein:

a) the methionine (M)-amino acid entity is L-methionine or a salt thereof; and

b) the cysteine (C)-amino acid entity is N-acetylcysteine.

60. The composition of embodiment 58, wherein the total wt. % of (a)-(b) is greater than the total wt. % of other amino acid entities in the composition.
61. A composition comprising:

a) an ornithine (Orn)-amino acid entity; and

b) an arginine (R)-amino acid entity;

provided that:

(i) at least one amino acid entity is not provided as a peptide of more than 20 amino acid residues in length;

(ii) the amino acid entities of (a)-(b) are selected from Table 2; and

(iii) the composition comprises fewer than 20 different amino acid entities.

62. The composition of embodiment 61, wherein:

a) the ornithine (Orn)-amino acid entity is ornithine or a salt thereof; and

b) the arginine (R)-amino acid entity is L-arginine.

63. The composition of embodiment 61, wherein the total wt. % of (a)-(b) is greater than the total wt. % of other amino acid entities in the composition.
64. The composition of any preceding embodiment, wherein the composition comprises fewer than 19, 18, 17, 16, 15, 14, 13, 12, 11, 10, 9, 8, 7, or 6 different amino acid entities (and optionally, at least 5 different amino acid entities).
65. The composition of any of the preceding embodiments, wherein the L-amino acid entity is chosen from the group consisting of L-leucine or a salt thereof, or a dipeptide or salt thereof, or tripeptide or salt thereof, comprising L-leucine; oxo-leucine; HMB (beta-hydroxy-beta-methylbutyrate); oxo-leucine; isovaleryl-CoA; D-Leucine; N-Acetyl-Leucine; or a combination thereof.
66. The composition of any of the preceding embodiments, wherein the I-amino acid entity is chosen from the group consisting of L-Isoleucine or a salt thereof, or a dipeptide or salt thereof, or tripeptide or salt thereof, comprising L-isoleucine; 2-Oxo-3-methyl-valerate; threonine; 2-Oxo-3-methyl-valerate; Methylbutyl-CoA; D-Isoleucine; N-Acetyl-Isoleucine; or a combination thereof.
67. The composition of any of the preceding embodiments, wherein the V-amino acid entity is chosen from the group consisting of L-valine or a salt thereof, or a dipeptide or salt thereof, or tripeptide or salt thereof, comprising L-valine; 2-Oxo-valerate; Isobutyl-CoA; 3-HIB-CoA; D-Valine; N-Acetyl-Valine; or a combination thereof.
68. The composition of any of the preceding embodiments, wherein the Q-amino acid entity is chosen from the group consisting of L-glutamine or a salt thereof, or a dipeptide or salt thereof, or tripeptide or salt thereof, comprising L-glutamine; glutamate; carbamoyl-P glutamate; D-glutamine; and N-acetylglutamine; or a combination thereof.
69. The composition of any of the preceding embodiments, wherein the E-amino acid entity is chosen from the group consisting of L-glutamate or a salt thereof, or a dipeptide or salt thereof, or tripeptide or salt thereof, comprising L-glutamate; 2-oxo-glutarate; glutathione; glutamine;

carbamoyl-P; or a combination thereof.

70. The composition of any of the preceding embodiments, wherein the D-amino acid entity is chosen from the group consisting of L-aspartate or a salt thereof, or a dipeptide or salt thereof, or tripeptide or salt thereof, comprising L-aspartate; fumarate; adenylosuccinate; or a combination thereof.
71. The composition of any of the preceding embodiments, wherein the G-amino acid entity is chosen from the group consisting of glycine or a salt thereof, or a dipeptide or salt thereof, or tripeptide or salt thereof, comprising glycine; glutathione; L-serine; or a combination thereof.
72. The composition of any of the preceding embodiments, wherein the S-amino acid entity is chosen from the group consisting of L-serine or a salt thereof, or a dipeptide or salt thereof, or tripeptide or salt thereof, comprising L-serine; phosphoserine; p-hydroxypyruvate; glycine; glycine; tryptophan; acetylserine; cystathionine; phosphatidylserine; or a combination thereof.
73. The composition of any of the preceding embodiments, wherein the M-amino acid entity is chosen from the group consisting of L-methionine or a salt thereof, or a dipeptide or salt thereof, or tripeptide or salt thereof, comprising L-methionine; homocysteine; L-cysteine; or a combination thereof.
74. The composition of any of the preceding embodiments, wherein the C-amino acid entity is chosen from the group consisting of N-Acetylcysteine or a salt thereof, or a dipeptide or salt thereof, or tripeptide or salt thereof, comprising N-Acetylcysteine; serine; acetylserine; cystathionine; glutathione; cystathionine; homocysteine; methionine; D-Cysteine; L-Cysteine; cystine; cysteamine; or a combination thereof.
75. The composition of any of the preceding embodiments, wherein the Orn-amino acid entity is chosen from the group consisting of L-ornithine or a salt thereof, or a dipeptide or salt thereof, or tripeptide or salt thereof, comprising L-ornithine; L-arginine; glycine; citrulline; ornithine α-ketoglutarate; ornithine HCl; or a combination thereof.
76. The composition of any of the preceding embodiments, wherein the R-amino acid entity is chosen from the group consisting of L-arginine or a salt thereof, or a dipeptide or salt thereof, or tripeptide or salt thereof, comprising L-arginine; ornithine; argininosuccinate; citrulline; aspartate; glutamate; agmatine; creatine; D-arginine; N-acetyl-arginine; or a combination thereof.
77. The composition of any of the preceding embodiments, wherein the H-amino acid entity is chosen from the group consisting of L-histidine or a salt thereof, or a dipeptide or salt thereof, or tripeptide or salt thereof, comprising L-histidine; histidinol; histidinal; ribose-5-phosphate; carnosine; histamine; urocanate; D-histidine; N-acetyl-histidine; or a combination thereof.
78. The composition of any of the preceding embodiments, wherein the Y-amino acid entity is chosen from the group consisting of L-tyrosine or a salt thereof, or a dipeptide or salt thereof, or tripeptide or salt thereof, comprising L-tyrosine; L-phenylalanine; or a combination thereof.
79. The composition of any of the preceding embodiments, wherein the W-amino acid entity is chosen from the group consisting of L-tryptophan or a salt thereof, or a dipeptide or salt thereof, or tripeptide or salt thereof, comprising L-tryptophan; L-serine; kynurenine; serotonin; or a combination thereof.
80. The composition of any one of the preceding embodiments, wherein when the composition is in powder form, at least 50 wt. % of the total wt. of the composition is one or more amino acid entities in free form.
81. A pharmaceutical composition comprising:

a) a composition of any one of embodiments 1-80; and

b) one or more pharmaceutically acceptable excipients;

provided that:

i) at least one amino acid entity is not provided as a peptide of more than 20 amino acid residues in length; and

ii) the amino acid entities in the composition are selected from Table 2.

82. A method for treating one or more symptoms selected from immobilization, malnutrition, fasting, aging, autophagy, reduced protein synthesis, anabolic resistance, junction integrity, insulin resistance, decreased mitochondrial biogenesis, decreased myogenesis or myotube growth, anaplerosis, or an energy deficit, wherein the method comprises administering to a subject in need thereof an effective amount of a composition from any one of embodiments 1-80;
provided that:

i) at least one amino acid entity is not provided as a peptide of more than 20 amino acid residues in length; and

ii) the amino acid entities in the composition are selected from Table 2.

83. A method for improving muscle function, wherein the method comprises administering to a subject in need thereof an effective amount of the composition of any one of embodiments 1-80; provided that:

i) at least one amino acid entity is not provided as a peptide of more than 20 amino acid residues in length; and

ii) the amino acid entities in the composition are selected from Table 2.

84. The method of embodiment 82 or 83, wherein the subject has a disease or disorder selected from the group consisting of a rare muscle disease, muscle atrophy, sarcopenia, muscle deterioration, muscle decay, cachexia, drug-induced myopathy, muscular dystrophy, myopenia, muscle weakness, perceived muscle weakness, ICU-acquired myopathy, burns-related myopathy, a neuromuscular disorder, ventilator-induced diaphragmatic dystrophy, hyponatremia, hypokalemia, a calcium deficiency, hypercalcemia, amyotrophic lateral sclerosis, and a bone weakness disease.
85. The method of any of embodiments 82-84, wherein the subject has or is identified as having decreased muscle function due to aging, injury, muscle atrophy, infection, disease, stroke, or a fracture or other trauma.
86. The method of any of embodiments 82-85, wherein the subject has had a rotator cuff surgery, knee surgery, hip surgery, joint replacement, injury repair surgery, or has worn a cast prior to administration of the composition.
87. A dietary composition comprising a composition of any one of embodiments 1-80 provided that:

i) at least one amino acid entity is not provided as a peptide of more than 20 amino acid residues in length; and

(ii) the amino acid entities in the composition are selected from Table 2.

88. The dietary composition of embodiment 87, wherein the dietary composition is chosen from a medical food, a functional food, or a supplement.
89. A method of providing amino acid entities to a subject comprising administering to the subject an effective amount of the composition of any one of embodiments 1-80;
provided that:

i) at least one amino acid entity is not provided as a peptide of more than 20 amino acid residues in length; and

(ii) the amino acid entities in the composition are selected from Table 2.

90. A method of manufacturing or making a composition comprising forming a composition comprising at least five of the following:

a) a leucine (L)-amino acid entity;

b) an isoleucine (I)-amino acid entity;

c) a valine (V)-amino acid entity;

d) a glutamine (Q)-amino acid entity;

e) a glutamic acid (E)-amino acid entity;

f) an aspartic acid (D)-amino acid entity;

g) a glycine (G)-amino acid entity;

h) a serine (S)-amino acid entity;

i) a methionine (M)-amino acid entity;

j) a cysteine (C)-amino acid entity;

k) an ornithine (Orn)-amino acid entity;

l) an arginine (R)-amino acid entity;

m) a histidine (H)-amino acid entity;

n) a tyrosine (Y)-amino acid entity; and

o) a tryptophan (W)-amino acid entity;

wherein:

i) at least one amino acid entity is not provided as a peptide of more than 20 amino acid residues in length; and

(ii) the amino acid entities of (a)-(o) are selected from Table 2.

91. A method for increasing myogenesis, wherein the method comprises administering to a subject in need thereof an effective amount of the composition of any one of embodiments 1-80;
provided that:

i) at least one amino acid entity is not provided as a peptide of more than 20 amino acid residues in length; and

ii) the amino acid entities in the composition are selected from Table 2.

92. A method for increasing muscle protein synthesis, wherein the method comprises administering to a subject in need thereof an effective amount of the composition of any one of embodiments 1-80;
provided that:

i) at least one amino acid entity is not provided as a peptide of more than 20 amino acid residues in length; and

ii) the amino acid entities in the composition are selected from Table 2.

93. A method for increasing muscle mass, wherein the method comprises administering to a subject in need thereof an effective amount of the composition of any one of embodiments 1-80;
provided that:

i) at least one amino acid entity is not provided as a peptide of more than 20 amino acid residues in length; and

ii) the amino acid entities in the composition are selected from Table 2.

94. A method for improving muscle quality, wherein the method comprises administering to a subject in need thereof an effective amount of the composition of any one of embodiments 1-80;
provided that:

i) at least one amino acid entity is not provided as a peptide of more than 20 amino acid residues in length; and

ii) the amino acid entities in the composition are selected from Table 2.

### EXAMPLES

The Examples below are set forth to aid in the understanding of the inventions, but is not intended to, and should not be construed to, limit its scope in any way.

### Example 1—In Vitro Model for Myogenesis

Muscle cell differentiation (myogenesis) is important for muscle growth and recovery from injury. Myogenesis is impaired in aging, muscular dystrophies, and other diseases with muscle wasting. The ability of amino acids to influence lipid accumulation in muscle cells was assessed using a modified version of MyoScreen (Cytoo SA, Grenoble, France).

### Cell Seeding and Differentiation

Primary myoblasts (W018) were amplified and seeded into CYTOOplates on Day 0. After one day differentiation was started in DMEM/F12+GlutaMAX supplemented with 0.1% Horse Serum.

### Culture Conditions and Different Amino Acids Combinations

After 4 days the differentiated myotubes were washed and culture conditions were changed to DMEM containing a profile of amino acids consistent with that found in healthy individuals at a 6.6-fold lower concentration than that found in human plasma (0.15×) (Wishart, D. S.; Tzur, D.; Knox, C., et al., Nucleic Acids Res. 2007, 35, D521-D526; Wishart, D. S.; Knox, C.; Guo, A. C., et al., Nucleic Acids Res. 2009, 37, D603-D610; Wishart, D. S.; Jewison, T., Guo, A. C.; Wilson, M.; Knox, C., et al., Nucleic Acids Res. 2013, 41, D801-D807; Wishart, D. S.; Feunang, Y. D.; Marcu, A.; Guo, A. C.; Liang. K., et al., Nucleic Acids Res. 2018, 46, D608-D617). Additionally, cells were untreated or treated with individual or combinations of amino acids at concentrations equal that found in human plasma (1×). Specific combinations of amino acids were derived by identification of differentially regulated amino acids at restrictive (0.15×) and permissive (1×) concentrations of all amino acids.

Fusion Index Analysis after 4 Days by Fluorescence Microscopy

After 4 days of culture cells were washed twice with PBS 1× (Gibco), fixed with 4% Paraformaldehyde, and washed twice with PBS 1× (100 μL) After fixation cells were immuno-stained for troponin to identify specific myotubes, and nuclei were stained with Hoechst 3342 (Life Technologies) diluted to 4 μg/mL. Images were acquired with Operetta HCS platform (Perkin Elmer) using a 10× objective in two fluorescent channels: nuclei, Troponin T. Image processing and analysis were performed with a dedicated algorithm developed on Acapella high Content imaging Software (Perkin Elmer) at CYTOO. Nuclei within myotubes were counted under myotube mask generated by troponin co-stain and fusion index was calculated as the percentage of fused nuclei in myotube over total nuclei. Statistical analysis was done with T-Test relative to the untreated condition.

### Results

### Fusion Index

Table 1.1 shows fusion index results of myotubes cultured under different profiles of amino acids, arranged by rank according to strongest effects.

TABLE 1.1
Fusion Index

| | | Fusion Index | |
|---|---|---|---|
| | | | standard |
| rank | Amino acid compositions | median | deviation |
| 1 | M + C | 0.36273029 | 0.0665288 |
| 2 | L + I + V + Q + E + D + G + | 0.30809112 | 0.10956064 |
| | S + C + Orn | | |
| 3 | C | 0.29591669 | 0.06960594 |
| 4 | M | 0.27601812 | 0.10091232 |
| 5 | L + I + V + E + D + S + C + H | 0.25903419 | 0.08590628 |
| 6 | L + I + V + E + D + G | 0.25386541 | 0.0891562 |
| 7 | L + + V + Om + R | 0.23650567 | 0.13576903 |
| 8 | L + + V + D + C + Or | 0.23527073 | 0.07974936 |
| 9 | Q + E | 0.22734616 | 0.05263674 |
| 10 | L + I + V + C + Orn | 0.22474338 | 0.10116666 |
| 11 | L + I | 0.20479275 | 0.08465307 |
| 12 | Orn + R | 0.20116434 | 0.09607172 |
| 13 | L + I + V + E + D + S + | 0.18966817 | 0.06900861 |
| | C + Orn + H | | |
| 14 | Q + E + D + G + Orn + R | 0.1878724 | 0.03779207 |
| 15 | L + I + V + D | 0.16782693 | 0.10225187 |
| 16 | 1X HMDB | 0.15366718 | 0.09069625 |
| 17 | L + I + V + D + M + C + | 0.15293364 | 0.0456839 |
| | R + H + Y + W | | |
| 18 | L + I + V + Q + E + D + | 0.14135549 | 0.02789584 |
| | G + S + M + C + Orn + | | |
| | R + H + Y + W | | |
| 19 | E + D + G | 0.13730881 | 0.07165649 |
| 20 | L + I + V + Q + G + S + | 0.12485815 | 0.10241871 |
| | M + C + R + H + Y + W | | |
| 21 | Orn | 0.10808525 | 0.06802871 |
| 22 | L + I + V + E + D + S + Orn + H | 0.10695924 | 0.05315784 |
| 23 | E | 0.10688135 | 0.07772593 |
| 24 | I | 0.10297649 | 0.06540957 |
| 25 | V | 0.1011398 | 0.07568096 |
| 26 | L | 0.08449174 | 0.09186725 |
| 27 | L + I + V | 0.0838836 | 0.05078389 |
| 28 | R | 0.08114453 | 0.09211575 |
| 29 | G | 0.06884141 | 0.09122918 |
| 30 | W | 0.06165096 | 0.11553702 |
| 31 | D | 0.05657125 | 0.06940974 |
| 32 | L + I + V + E + D + S + C + | 0.05620463 | 0.06212136 |
| | Orn + H + Y + W | | |
| 33 | Q + E + D + G + S | 0.05320978 | 0.05468235 |
| 34 | R + H + Y + W | 0.05296224 | 0.05611982 |
| 35 | L + I + V + M + C | 0.03348832 | 0.10837856 |
| 36 | Q | 0.01063277 | 0.08806687 |
| 37 | .15X HMDB | 0.00327753 | 0.08235713 |
| 38 | s | −0.0003011 | 0.06895957 |
| 39 | L + I + V + Om | −0.0415812 | 0.09687396 |
| 40 | Y | −0.0520584 | 0.09657046 |
| 41 | L + I + V + M + C + R + | −0.0655261 | 0.05632666 |
| | H + Y + W | | |
| 42 | H | −0.1733657 | 0.1806447 |

Table 1.1 summarizes myogenesis (myotube differentiation) data experiments for each treatment group for increasing a measure of myogenesis, fusion index. Consistently, specific combinations of amino acids (such as M+C, L+I+V+Q+E+D+G+S+C+Orn, L+I+V+E+D+S+C+H, L+I+V+E+D+G, L+I+V+Orn+R, L+I+V+D+C+Orn, Q+E, L+I+V+C+Orn, Orn+R, L+I+V+E+D+S+C+Orn+H, Q+E+D+G+Orn+R, L+I+V+D, L+I+V+D+M+C+R+H+Y+W, L+I+V+Q+E+D+G+S+M+C+Orn+R+H+Y+W, E+D+G, L+I+V+Q+G+S+M+C+R+H+Y+W, and L+I+V+E+D+S+Orn+H) increased fusion index in differentiating cultured human myotubes, much more than other compositions.

### Example 2—In Vitro Model for Muscle Protein Synthesis

Muscle protein synthesis is important for muscle growth and muscle protein breakdown drives muscle loss. Increasing muscle protein synthesis remains an important consideration in diseases with muscle wasting in order to counteract the deleterious protein breakdown. Defective protein synthesis (anabolic resistance) is a hallmark of aging and complex diseases like cirrhosis, chronic kidney disease, and COPD. The ability of amino acids to influence protein synthesis in muscle cells was assessed using a modified version of MyoScreen (Cytoo SA, Grenoble Fr).

### Cell Seeding and Differentiation

Primary myoblasts (W018) were amplified and seeded into CYTOOplates on Day 0. After one day differentiation was started in DMEM/F12+GlutaMAX supplemented with 0.1% Horse Serum.

### Culture Conditions and Different Amino Acids Combinations

After 4 days the differentiated myotubes were washed and culture conditions were changed to DMEM containing a profile of amino acids consistent with that found in healthy individuals at a 6.6-fold lower concentration than that found in human plasma (0.15×) (Wishart, D. S.; Tzur, D.; Knox, C., et al., Nucleic Acids Res. 2007, 35, D521-D526; Wishart, D. S.; Knox, C.; Guo, A. C., et al., Nucleic Acids Res. 2009, 37, D603-D610; Wishart, D. S.; Jewison, T., Guo, A. C.; Wilson, M.; Knox, C., et al., Nucleic Acids Res. 2013, 41, D801-D807; Wishart, D. S.; Feunang, Y. D.; Marcu, A.; Guo, A. C.; Liang. K., et al., Nucleic Acids Res. 2018, 46, D608-D617). Additionally, cells were untreated or treated with individual or combinations of amino acids at concentrations equal that found in human plasma (1×). Specific combinations of amino acids were derived by identification of differentially regulated amino acids at restrictive (0.15×) and permissive (1×) concentrations of all amino acids.

Protein Synthesis after 4 Days by Fluorescence Microscopy

After 4 days of culture, cells were washed twice with PBS 1× (Gibco), fixed with 4% Paraformaldehyde, and washed twice with PBS 1× (100 μL). Cells were then permeabilized by 0.5% Triton X-100 followed by three washes with PBS. After fixation, cells were immuno-stained for troponin to identify specific myotubes. Detection of protein synthesis was achieved using Click-iT™ Plus OPP Alexa Fluor™ 488 diluted to 10 μM. In the Click-iT assay, 0-propargyl-puromycin (OPP) is efficiently incorporated into newly translated proteins in vivo for 30 minutes and then fluorescently labeled with Alexa Fluor® dye, which can be detected by high-content imaging (Molecular Devices). Click-iT signal was restricted to the myotube mask generated by the troponin co-stain. Data was collected as Click-iT mean intensity in myotubes (au). Images were acquired with Operetta HCS platform (Perkin Elmer) using a 10× objective in three fluorescent channels: nuclei, Troponin T, and Click-iT. Image processing and analysis were performed with a dedicated algorithm developed on Acapella high Content imaging Software (Perkin Elmer) at CYTOO. Statistical analysis was done with T-Test relative to the untreated condition.

### Results

### Protein Synthesis

Table 2.1 shows of myotubes cultured under different profiles of amino acids, arranged by rank according to strongest effects on protein synthesis

TABLE 2.1
Protein Synthesis

| | | | standard |
|---|---|---|---|
| rank | Amino acid compositions | median | deviation |
| 1 | L + I + V + D + M + C + | 0.26559152 | 0.074459698 |
| | R + H + Y + W | | |
| 2 | L + I + V + Q + E + D + | 0.241968571 | 0.051457537 |
| | G + S + M + C + Orn + | | |
| | R + H + Y + W | | |
| 3 | L + I + V + E + D + | 0.233291027 | 0.05301068 |
| | S + C + H | | |
| 4 | L + I + V + M + C + | 0.216688292 | 0.034219812 |
| | R + H + Y + W | | |
| 5 | L + I + V + E + D + | 0.210782015 | 0.022969702 |
| | S + C + Orn + H | | |
| 6 | L + I + V + D + C + Om | 0.200438088 | 0.05952043 |
| 7 | L + I + V + E + D + S + | 0.169601247 | 0.024732295 |
| | C + Orn + H + Y + W | | |
| 8 | L + I + V | 0.158609744 | 0.053246271 |
| 9 | L + I + V + Q + G + S + | 0.14214476 | 0.127470127 |
| | M + C + R + H + Y + W | | |
| 10 | 1X HMDB | 0.129919053 | 0.063630651 |
| 11 | Q + E + D + G + S | 0.128967789 | 0.093453305 |
| 12 | L + + V + Om + R | 0.127036146 | 0.057809915 |
| 13 | L + I + V + E + D + | 0.125172054 | 0.050523325 |
| | S + Orn + H | | |
| 14 | L + I + V + M + C | 0.122484485 | 0.054762899 |
| 15 | L + I + V + Q + E + D + | 0.118047422 | 0.070748423 |
| | G + S + C + Orn | | |
| 16 | Q | 0.108291192 | 0.042271121 |
| 17 | D | 0.10784258 | 0.033254147 |
| 18 | E | 0.107389157 | 0.035057578 |
| 19 | L | 0.105328078 | 0.070883467 |
| 20 | R | 0.100501107 | 0.061204982 |
| 21 | Q + E + D + G + Orn + R | 0.090173146 | 0.056296319 |
| 22 | L + I | 0.087422682 | 0.03496249 |
| 23 | L + I + V + C + Orn | 0.084274264 | 0.089324466 |
| 24 | M + C | 0.079833351 | 0.065783565 |
| 25 | V | 0.062365791 | 0.060349624 |
| 26 | s | 0.046974141 | 0.043849887 |
| 27 | L + I + V + Orn | 0.042698323 | 0.05525421 |
| 28 | C | 0.037448369 | 0.097490015 |
| 29 | Y | 0.030363688 | 0.032352434 |
| 30 | Orn + R | 0.019937827 | 0.059119742 |
| 31 | G | 0.016734793 | 0.046467339 |
| 32 | .15X HMDB | 0.011144381 | 0.048008983 |
| 33 | 1 | 0.004077572 | 0.048418897 |
| 34 | H | 0.002706598 | 0.072838164 |
| 35 | L + I + V + D | −0.01193283 | 0.035476791 |
| 36 | Orn | 0.017425798 | 0.058570224 |
| 37 | L + I + V + E + D + G | 0.023568222 | 0.042540454 |
| 38 | R + H + Y + W | 0.030863924 | 0.064212923 |
| 39 | E + D + G | 0.041454616 | 0.067182985 |
| 40 | M | 0.050205392 | 0.047568395 |
| 41 | W | 0.064938237 | 0.062917665 |
| 42 | Q + E | 0.091245205 | 0.06245414 |

Table 2.1 summarizes muscle protein synthesis stimulation data showing results for each treatment group as described above. Surprisingly, specific combinations of amino acids (such as L+I+V+D+M+C+R+H+Y+W, L+I+V+Q+E+D+G+S+M+C+Orn+R+H+Y+W, L+I+V+E+D+S+C+H, L+I+V+M+C+R+H+Y+W, L+I+V+E+D+S+C+Orn+H, L+I+V+D+C+Orn, L+I+V+E+D+S+C+Orn+H+Y+W, L+I+V+Q+G+S+M+C+R+H+Y+W, Q+E+D+G+S, L+I+V+Orn+R, L+I+V+E+D+S+Orn+H, L+I+V+M+C, L+I+V+Q+E+D+G+S+C+Orn, Q+E+D+G+Orn+R, and L+I+V+C+Orn) increased protein synthesis in cultured human myotubes, much more than other compositions.

### SUMMARY OF EXAMPLES

As shown above, specific combinations of amino acids can be determined that have beneficial effects on i) protein synthesis, ii) myotube fusion/differentiation (or myogenesis), or iii) both phenotypes. Table 3.1 shows the top 25 amino acid compositions tested ranked by their effects on each phenotype separately, as well as the top 25 amino acid compositions tested ranked by their combined effects on both protein synthesis and myogenesis (with weighting of 2× protein synthesis and 1× for fusion index).

Muscle diseases are complex and driven by a multitude of unique mechanisms. Recovery from muscle loss or injury requires coordination of many biological, cellular, and molecular processes. The amino acid compositions defined herein are designed to promote muscle growth and function for a wide range of muscle pathologies. The amino acid compositions disclosed in this application are able to promote protein synthesis, muscle cell differentiation, or both, whereas other compositions, are only able to influence some, but not all of those important processes required for maintaining muscle health.

In particular, specific combinations of amino acids (such as L+I+V+E+D+S+C+H, L+I+V+D+M+C+R+H+Y+W, L+I+V+D+C+Orn, L+I+V+Q+E+D+G+S+M+C+Orn+R+H+Y+W, L+I+V+E+D+S+C+Orn+H, L+I+V+Orn+R, L+I+V+Q+E+D+G+S+C+Orn, L+I+V+Q+G+S+M+C+R+H+Y+W, L+I+V+E+D+S+C+Orn+H+Y+W, L+I+V+E+D+S+Orn+H, L+I+V+M+C+R+H+Y+W, M+C, Q+E+D+G+S, L+I+V+C+Orn, Q+E+D+G+Orn+R and L+I+V+M+C) performed well in both protein synthesis and myogenesis/myotube differentiation, indicating their particular utility in muscle health and disease applications.

TABLE 3.1
Summary of top ranking of amino acid compositions across phenotypes

| | | | combined rank |
|---|---|---|---|
| rank | protein synthesis | fusion index | (2ps + 1fi) |
| 1 | L + I + V + | M + C | L + I + V + E + D + |
| | D + M + C + | | S + C + H |
| | R + H + Y + W | | |
| 2 | L + I + V + | L + I + V + | L + I + V + D + M + |
| | Q + E + D + | Q + E + D + G + | C + R + H + Y + W |
| | G + S + M + C + | S + C + Orn | |
| | Orn + R + | | |
| | H + Y + W | | |
| 3 | L + I + V + E + D + | C | L + I + V + D + |
| | S + C + H | | C + Orn |
| 4 | L + I + V + M + C + | M | L + I + V + Q + |
| | R + H + Y + W | | E + D + G + |
| | | | S + M + C + Orn + |
| | | | R + H + Y + W |
| 5 | L + I + V + E + D + | L + I + V + E + | L + I + V + E + D + |
| | S + C + Orn + H | D + S + C + H | S + C + Orn + H |
| 6 | L + I + V + | L + I + V + E + | L + I + V + Orn + R |
| | D + C + Orn | D + G | |
| 7 | L + I + V + E + | L + I + V + | L + I + V + Q + |
| | D + S + C + Orn + | Orn + R | E + D + G + |
| | H + Y + W | | S + C + Orn |
| 8 | L + I + V | L + I + V + D + | 1X HMDB |
| | | C + Orn | |
| 9 | L + I + V + Q + | Q + E | L + I + V + Q + |
| | G + S + M + C + | | G + S + M + C + |
| | R + H + Y + W | | R + H + Y + W |
| 10 | 1X HMDB | L + I + V + | L + I + V |
| | | C + Orn | |
| 11 | Q + E + D + G + S | L + I | L + I + V + E + |
| | | | D + S + C + Orn + |
| | | | H + Y + W |
| 12 | L + I + V + Orn + R | Orn + R | L + I + V + E + D + |
| | | | S + Orn + H |
| 13 | L + I + V + E + D + | L + I + V + E + D + | L + I + V + M + C + |
| | S + Orn + H | S + C + Orn + H | R + H + Y + W |
| 14 | L + I + V + M + C | Q + E + D + G + | M + C |
| | | Orn + R | |
| 15 | L + I + V + Q + E + | L + I + V + D | L + I |
| | D + G + S + C + Orn | | |
| 16 | Q | 1X HMDB | Q + E + D + G + S |
| 17 | D | L + I + V + D + M + | L + I + V + C + Orn |
| | | C + R + H + Y + W | |
| 18 | E | L + I + V + Q + | Q + E + D + G + |
| | | E + D + G + S + | Orn + R |
| | | M + C + Orn + | |
| | | R + H + Y + W | |
| 19 | L | E + D + G | C |
| 20 | R | L + I + V + Q + | E |
| | | G + S + M + C + | |
| | | R + H + Y + W | |
| 21 | Q + E + D + G + | Orn | L + I + V + M + C |
| | Orn + R | | |
| 22 | L + I | L + I + V + E + D + | L |
| | | S + Orn + H | |
| 23 | L + I + V + C + Orn | E | D |
| 24 | M + C | I | Q |
| 25 | V | V | R |

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

What is claimed is:

1. A composition comprising:
a) a leucine (L)-amino acid entity selected from L-leucine or a salt thereof;
b) an isoleucine (I)-amino acid entity selected from L-isoleucine or a salt thereof;
c) a valine (V)-amino acid entity selected from L-valine or a salt thereof;
d) an aspartic acid (D)-amino acid entity selected from L-aspartate or a salt thereof;
e) a methionine (M)-amino acid entity selected from L-methionine or a salt thereof;
f) a cysteine (C)-amino acid entity selected from N-acetylcysteine or a salt thereof;
g) an arginine (R)-amino acid entity selected from L-arginine or a salt thereof;
h) a histidine (H)-amino acid entity selected from L-histidine or a salt thereof;
i) a tyrosine (Y)-amino acid entity selected from L-tyrosine or a salt thereof; and
j) a tryptophan (W)-amino acid entity selected from L-tryptophan or a salt thereof,
provided that:
(i) at least one amino acid entity is not provided as a peptide of more than 20 amino acid residues in length;
(ii) the amino acid entities of (a)-(j) are selected from Table 2; and
(iii) the composition comprises fewer than 20 different amino acid entities.

2. A composition comprising:
a) a leucine (L)-amino acid entity selected from L-leucine or a salt thereof;
b) an isoleucine (I)-amino acid entity selected from L-isoleucine or a salt thereof;
c) a valine (V)-amino acid entity selected from L-valine or a salt thereof;
d) a glutamine (Q)-amino acid entity selected from L-glutamine or a salt thereof;
e) a glutamic acid (E)-amino acid entity selected from L-glutamate or a salt thereof;
f) an aspartic acid (D)-amino acid entity selected from L-aspartate or a salt thereof;
g) a glycine (G)-amino acid entity selected from glycine or a salt thereof;
h) a serine (S)-amino acid entity selected from L-serine or a salt thereof;
i) a methionine (M)-amino acid entity selected from L-methionine or a salt thereof;
j) a cysteine (C)-amino acid entity selected from N-acetylcysteine or a salt thereof;
k) an ornithine (Orn)-amino acid entity selected from L-ornithine or a salt thereof;
l) an arginine (R)-amino acid entity selected from L-arginine or a salt thereof;
m) a histidine (H)-amino acid entity selected from L-histidine or a salt thereof;
n) a tyrosine (Y)-amino acid entity selected from L-tyrosine or a salt thereof; and
o) a tryptophan (W)-amino acid entity selected from L-tryptophan or a salt thereof,
provided that:
(i) at least one amino acid entity is not provided as a peptide of more than 20 amino acid residues in length;
(ii) the amino acid entities of (a)-(o) are selected from Table 2; and
(iii) the composition comprises fewer than 20 different amino acid entities.

3. A composition comprising:
a) a leucine (L)-amino acid entity selected from L-leucine or a salt thereof;
b) an isoleucine (I)-amino acid entity selected from L-isoleucine or a salt thereof;
c) a valine (V)-amino acid entity selected from L-valine or a salt thereof;
d) a glutamic acid (E)-amino acid entity selected from L-glutamate or a salt thereof;
e) an aspartic acid (D)-amino acid entity selected from L-aspartate or a salt thereof;
f) a serine (S)-amino acid entity selected from L-serine or a salt thereof;
g) a cysteine (C)-amino acid entity selected from N-acetylcysteine or a salt thereof; and
h) a histidine (H)-amino acid entity selected from L-histidine or a salt thereof,
provided that:
(i) at least one amino acid entity is not provided as a peptide of more than 20 amino acid residues in length;
(ii) the amino acid entities of (a)-(h) are selected from Table 2; and
(iii) the composition comprises fewer than 20 different amino acid entities.

4. A composition comprising:
a) a leucine (L)-amino acid entity selected from L-leucine or a salt thereof;
b) an isoleucine (I)-amino acid entity selected from L-isoleucine or a salt thereof;
c) a valine (V)-amino acid entity selected from L-valine or a salt thereof;
d) a methionine (M)-amino acid entity selected from L-methionine or a salt thereof;
e) a cysteine (C)-amino acid entity selected from N-acetylcysteine or a salt thereof;
f) an arginine (R)-amino acid entity selected from L-arginine or a salt thereof;
g) a histidine (H)-amino acid entity selected from L-histidine or a salt thereof;
h) a tyrosine (Y)-amino acid entity selected from L-tyrosine or a salt thereof; and
i) a tryptophan (W)-amino acid entity selected from L-tryptophan or a salt thereof,
provided that:
(i) at least one amino acid entity is not provided as a peptide of more than 20 amino acid residues in length;
(ii) the amino acid entities of (a)-(i) are selected from Table 2; and
(iii) a composition comprises fewer than 20 different amino acid entities.

5. A composition comprising:
a) a leucine (L)-amino acid entity selected from L-leucine or a salt thereof;
b) an isoleucine (I)-amino acid entity selected from L-isoleucine or a salt thereof;
c) a valine (V)-amino acid entity selected from L-valine or a salt thereof;
d) a glutamic acid (E)-amino acid entity selected from L-glutamate or a salt thereof;
e) an aspartic acid (D)-amino acid entity selected from L-aspartate or a salt thereof;
f) a serine (S)-amino acid entity selected from L-serine or a salt thereof;
g) a cysteine (C)-amino acid entity selected from N-acetylcysteine or a salt thereof;
h) an ornithine (Orn)-amino acid entity selected from L-ornithine or a salt thereof; and
i) a histidine (H)-amino acid entity selected from L-histidine or a salt thereof,
provided that:
(i) at least one amino acid entity is not provided as a peptide of more than 20 amino acid residues in length;
(ii) the amino acid entities of (a)-(i) are selected from Table 2; and
(iii) the composition comprises fewer than 20 different amino acid entities.

6. A composition comprising:
a) a leucine (L)-amino acid entity selected from L-leucine or a salt thereof;
b) an isoleucine (I)-amino acid entity selected from L-isoleucine or a salt thereof;
c) a valine (V)-amino acid entity selected from L-valine or a salt thereof;
d) an aspartic acid (D)-amino acid entity selected from L-aspartate or a salt thereof;
e) a cysteine (C)-amino acid entity selected from N-acetylcysteine or a salt thereof; and
f) an ornithine (Orn)-amino acid entity selected from L-ornithine or a salt thereof,
provided that:
(i) at least one amino acid entity is not provided as a peptide of more than 20 amino acid residues in length;
(ii) the amino acid entities of (a)-(f) are selected from Table 2; and
(iii) the composition comprises fewer than 20 different amino acid entities.

7. A composition comprising:
a) a leucine (L)-amino acid entity selected from L-leucine or a salt thereof;
b) an isoleucine (I)-amino acid entity selected from L-isoleucine or a salt thereof;
c) a valine (V)-amino acid entity selected from L-valine or a salt thereof;
d) a glutamic acid (E)-amino acid entity selected from L-glutamate or a salt thereof;
e) an aspartic acid (D)-amino acid entity selected from L-aspartate or a salt thereof;
f) a serine (S)-amino acid entity selected from L-serine or a salt thereof;
g) a cysteine (C)-amino acid entity selected from N-acetylcysteine or a salt thereof;
h) an ornithine (Orn)-amino acid entity selected from L-ornithine or a salt thereof;
i) a histidine (H)-amino acid entity selected from L-histidine or a salt thereof;
j) a tyrosine (Y)-amino acid entity selected from L-tyrosine or a salt thereof; and
k) a tryptophan (W)-amino acid entity selected from L-tryptophan or a salt thereof,
provided that:
(i) at least one amino acid entity is not provided as a peptide of more than 20 amino acid residues in length;
(ii) the amino acid entities of (a)-(k) are selected from Table 2; and
(iii) the composition comprises fewer than 20 different amino acid entities.

8. A composition comprising:
a) a leucine (L)-amino acid entity selected from L-leucine or a salt thereof;
b) an isoleucine (I)-amino acid entity selected from L-isoleucine or a salt thereof;
c) a valine (V)-amino acid entity selected from L-valine or a salt thereof;
d) a glutamine (Q)-amino acid entity selected from L-glutamine or a salt thereof;
e) a glycine (G)-amino acid entity selected from Glycine or a salt thereof;
f) a serine (S)-amino acid entity selected from L-serine or a salt thereof;
g) a methionine (M)-amino acid entity selected from L-methionine or a salt thereof;
h) a cysteine (C)-amino acid entity selected from N-acetylcysteine or a salt thereof;
i) an arginine (R)-amino acid entity selected from L-arginine or a salt thereof;
j) a histidine (H)-amino acid entity selected from L-histidine or a salt thereof;
k) a tyrosine (Y)-amino acid entity selected from L-tyrosine or a salt thereof; and
l) a tryptophan (W)-amino acid entity selected from L-tryptophan or a salt thereof,
provided that:
(i) at least one amino acid entity is not provided as a peptide of more than 20 amino acid residues in length;
(ii) the amino acid entities of (a)-(l) are selected from Table 2; and
(iii) the composition comprises fewer than 20 different amino acid entities.

9. A composition comprising:
a) a glutamine (Q)-amino acid entity selected from L-glutamine or a salt thereof;
b) a glutamic acid (E)-amino acid entity selected from L-glutamate or a salt thereof;
c) a aspartic acid (D)-amino acid entity selected from L-aspartate or a salt thereof;
d) a glycine (G)-amino acid entity selected from Glycine or a salt thereof; and
e) a serine (S)-amino acid entity selected from L-serine or a salt thereof,
provided that:
(i) at least one amino acid entity is not provided as a peptide of more than 20 amino acid residues in length;
(ii) the amino acid entities of (a)-(e) are selected from Table 2; and
(iii) the composition comprises fewer than 20 different amino acid entities.

10. A composition comprising:
a) a leucine (L)-amino acid entity selected from L-leucine or a salt thereof;
b) an isoleucine (I)-amino acid entity selected from L-isoleucine or a salt thereof;
c) a valine (V)-amino acid entity selected from L-valine or a salt thereof;
d) an ornithine (Orn)-amino acid entity selected from L-ornithine or a salt thereof; and
e) an arginine (R)-amino acid entity selected from L-arginine or a salt thereof,
provided that:
(i) at least one amino acid entity is not provided as a peptide of more than 20 amino acid residues in length;
(ii) the amino acid entities of (a)-(e) are selected from Table 2; and
(iii) the composition comprises fewer than 20 different amino acid entities.

11. A composition comprising:
a) a leucine (L)-amino acid entity selected from L-leucine or a salt thereof;
b) an isoleucine (I)-amino acid entity selected from L-isoleucine or a salt thereof;
c) a valine (V)-amino acid entity selected from L-valine or a salt thereof;
d) a glutamic acid (E)-amino acid entity selected from L-glutamate or a salt thereof;
e) an aspartic acid (D)-amino acid entity selected from L-aspartate or a salt thereof;
f) a serine (S)-amino acid entity selected from L-serine or a salt thereof;
g) an ornithine (Orn)-amino acid entity selected from L-ornithine or a salt thereof; and
h) a histidine (H)-amino acid entity selected from L-histidine or a salt thereof,
provided that:
(i) at least one amino acid entity is not provided as a peptide of more than 20 amino acid residues in length;
(ii) the amino acid entities of (a)-(h) are selected from Table 2; and
(iii) the composition comprises fewer than 20 different amino acid entities.

12. A composition comprising:
a) a leucine (L)-amino acid entity selected from L-leucine or a salt thereof;
b) an isoleucine (I)-amino acid entity selected from L-isoleucine or a salt thereof;
c) a valine (V)-amino acid entity selected from L-valine or a salt thereof;
d) a methionine (M)-amino acid entity selected from L-methionine or a salt thereof; and
e) a cysteine (C)-amino acid entity selected from N-acetylcysteine or a salt thereof,
provided that:
(i) at least one amino acid entity is not provided as a peptide of more than 20 amino acid residues in length;
(ii) the amino acid entities of (a)-(e) are selected from Table 2; and
(iii) the composition comprises fewer than 20 different amino acid entities.

13. A composition comprising:
a) a leucine (L)-amino acid entity selected from L-leucine or a salt thereof;
b) an isoleucine (I)-amino acid entity selected from L-isoleucine or a salt thereof;
c) a valine (V)-amino acid entity selected from L-valine or a salt thereof;
d) a glutamine (Q)-amino acid entity selected from L-glutamine or a salt thereof;
e) a glutamic acid (E)-amino acid entity selected from L-glutamate or a salt thereof;
f) an aspartic acid (D)-amino acid entity selected from L-aspartate or a salt thereof;
g) a glycine (G)-amino acid entity selected from Glycine or a salt thereof;
h) a serine (S)-amino acid entity selected from L-serine or a salt thereof;
i) a cysteine (C)-amino acid entity selected from N-acetylcysteine or a salt thereof; and
j) an ornithine (Orn)-amino acid entity selected from L-ornithine or a salt thereof,
provided that:
(i) at least one amino acid entity is not provided as a peptide of more than 20 amino acid residues in length;
(ii) the amino acid entities of (a)-(j) are selected from Table 2; and
(iii) the composition comprises fewer than 20 different amino acid entities.

14. A composition comprising:
a) a glutamine (Q)-amino acid entity selected from L-glutamine or a salt thereof;
b) a glutamic acid (E)-amino acid entity selected from L-glutamate or a salt thereof;
c) an aspartic acid (D)-amino acid entity selected from L-aspartate or a salt thereof;
d) a glycine (G)-amino acid entity selected from glycine or a salt thereof;
e) an ornithine (Orn)-amino acid entity selected from L-ornithine or a salt thereof; and
f) an arginine (R)-amino acid entity selected from L-arginine or a salt thereof,
provided that:
(i) at least one amino acid entity is not provided as a peptide of more than 20 amino acid residues in length;
(ii) the amino acid entities of (a)-(f) are selected from Table 2; and
(iii) the composition comprises fewer than 20 different amino acid entities.

15. A composition comprising:
a) a leucine (L)-amino acid entity selected from L-leucine or a salt thereof;
b) a isoleucine (I)-amino acid entity selected from L-isoleucine or a salt thereof;
c) a valine (V)-amino acid entity selected from L-valine or a salt thereof;
d) a cysteine (C)-amino acid entity selected from N-acetylcysteine or a salt thereof; and
e) a histidine (H)-amino acid entity selected from L-histidine or a salt thereof,
provided that:
(i) at least one amino acid entity is not provided as a peptide of more than 20 amino acid residues in length;
(ii) the amino acid entities of (a)-(e) are selected from Table 2; and
(iii) the composition comprises fewer than 20 different amino acid entities.

16. A composition comprising:
a) a leucine (L)-amino acid entity selected from L-leucine or a salt thereof;
b) an isoleucine (I)-amino acid entity selected from L-isoleucine or a salt thereof;
c) a valine (V)-amino acid entity selected from L-valine or a salt thereof;
d) a glutamic acid (E)-amino acid entity selected from L-glutamate or a salt thereof;
e) an aspartic acid (D)-amino acid entity selected from L-aspartate or a salt thereof; and
f) a glycine (G)-amino acid entity selected from glycine or a salt thereof,
provided that:
(i) at least one amino acid entity is not provided as a peptide of more than 20 amino acid residues in length;
(ii) the amino acid entities of (a)-(f) are selected from Table 2; and
(iii) the composition comprises fewer than 20 different amino acid entities.

17. A composition comprising:
a) a leucine (L)-amino acid entity selected from L-leucine or a salt thereof;
b) an isoleucine (I)-amino acid entity selected from L-isoleucine or a salt thereof;
c) a valine (V)-amino acid entity selected from L-valine or a salt thereof;
d) a cysteine (C)-amino acid entity or NAC entity selected from N-acetylcysteine; and
e) an ornithine (Orn)-amino acid entity selected from ornithine or a salt thereof,
provided that:
(i) at least one amino acid entity is not provided as a peptide of more than 20 amino acid residues in length;
(ii) the amino acid entities of (a)-(e) are selected from Table 2; and
(iii) the composition comprises fewer than 20 different amino acid entities.

18. A composition comprising:
a) a glutamic acid (E)-amino acid entity selected from L-glutamate or a salt thereof;
b) an aspartic acid (D)-amino acid entity selected from L-aspartate or a salt thereof; and
c) a glycine (G)-amino acid entity selected from glycine or a salt thereof,
provided that:
(i) at least one amino acid entity is not provided as a peptide of more than 20 amino acid residues in length;
(ii) the amino acid entities of (a)-(c) are selected from Table 2; and
(iii) the composition comprises fewer than 20 different amino acid entities.

19. A composition comprising:
a) a glutamine (Q)-amino acid entity selected from L-glutamine or a salt thereof; and
b) a glutamic acid (E)-amino acid entity selected from L-glutamic acid or a salt thereof,
provided that:
(i) at least one amino acid entity is not provided as a peptide of more than 20 amino acid residues in length;
(ii) the amino acid entities of (a)-(b) are selected from Table 2; and
(iii) the composition comprises fewer than 20 different amino acid entities.

20. A composition comprising:
a) a methionine (M)-amino acid entity selected from L-methionine or a salt thereof; and
b) a cysteine (C)-amino acid entity or a NAC entity comprising N-acetylcysteine,
provided that:
(i) at least one amino acid entity is not provided as a peptide of more than 20 amino acid residues in length;
(ii) the amino acid entities of (a)-(b) are selected from Table 2; and
(iii) the composition comprises fewer than 20 different amino acid entities.

21. A composition comprising:
a) an ornithine (Orn)-amino acid entity selected from ornithine or a salt thereof; and
b) an arginine (R)-amino acid entity comprising L-arginine.
provided that:
(i) at least one amino acid entity is not provided as a peptide of more than 20 amino acid residues in length;
(ii) the amino acid entities of (a)-(b) are selected from Table 2; and
(iii) the composition comprises fewer than 20 different amino acid entities.

22. A pharmaceutical composition comprising:
a) a composition of any one of claims 1-21; and
b) one or more pharmaceutically acceptable excipients;
provided that:
i) at least one amino acid entity is not provided as a peptide of more than 20 amino acid residues in length; and
ii) the amino acid entities in the composition are selected from Table 2.

23. A method for treating one or more symptoms selected from immobilization, malnutrition, fasting, aging, autophagy, reduced protein synthesis, anabolic resistance, junction integrity, insulin resistance, decreased mitochondrial biogenesis, decreased myogenesis or myotube growth, anaplerosis, or an energy deficit, wherein the method comprises administering to a subject in need thereof an effective amount of a composition from any one of claims 1-21;
provided that:
i) at least one amino acid entity is not provided as a peptide of more than 20 amino acid residues in length; and
ii) the amino acid entities in the composition are selected from Table 2.

24. A method for improving muscle function, wherein the method comprises administering to a subject in need thereof an effective amount of the composition of any one of claims 1-21;
provided that:
i) at least one amino acid entity is not provided as a peptide of more than 20 amino acid residues in length; and
ii) the amino acid entities in the composition are selected from Table 2.

25. A dietary composition comprising a composition of any one of claim 1-21 provided that:
i) at least one amino acid entity is not provided as a peptide of more than 20 amino acid residues in length; and
(ii) the amino acid entities in the composition are selected from Table 2.

26. A method of providing amino acid entities to a subject comprising administering to the subject an effective amount of the composition of any one of claims 1-21;
provided that:
i) at least one amino acid entity is not provided as a peptide of more than 20 amino acid residues in length; and
(ii) the amino acid entities in the composition are selected from Table 2.

27. A method for increasing myogenesis, wherein the method comprises administering to a subject in need thereof an effective amount of the composition of any one of claims 1-21;
provided that:
i) at least one amino acid entity is not provided as a peptide of more than 20 amino acid residues in length; and
ii) the amino acid entities in the composition are selected from Table 2.

28. A method for increasing muscle protein synthesis, wherein the method comprises administering to a subject in need thereof an effective amount of the composition of any one of claims 1-21;
provided that:
i) at least one amino acid entity is not provided as a peptide of more than 20 amino acid residues in length; and
ii) the amino acid entities in the composition are selected from Table 2.

29. A method for increasing muscle mass, wherein the method comprises administering to a subject in need thereof an effective amount of the composition of any one of claims 1-21;
provided that:
i) at least one amino acid entity is not provided as a peptide of more than 20 amino acid residues in length; and
ii) the amino acid entities in the composition are selected from Table 2.

30. A method for improving muscle quality, wherein the method comprises administering to a subject in need thereof an effective amount of the composition of any one of claims 1-21;
provided that:
i) at least one amino acid entity is not provided as a peptide of more than 20 amino acid residues in length; and
ii) the amino acid entities in the composition are selected from Table 2.
